(19) United States
(12) Patent Application Publication
(10) Pub. No.: US 2025/0161033 A1
(43) Pub. Date: May 22, 2025

(54) POLYMERIC HEART VALVE SYSTEM AND METHODS OF MAKING AND USING THEREOF

(71) Applicants: Arizona Board of Regents on Behalf of The University of Arizona, Tucson, AZ (US); The Research Foundation for The State University of New York, Albany, NY (US)

(72) Inventors: Marvin J. Slepian, Tucson, AZ (US); Danny Bluestein, Stony Brook, NY (US); Oren Rotman, Stony Brook, NY (US); Brandon J. Kovarovic, Stony Brook, NY (US)

(21) Appl. No.: 18/842,727

(22) PCT Filed: Mar. 1, 2023
(86) PCT No.: PCT/US2023/063483

§ 371 Date: Aug. 29, 2024

Related U.S. Application Data
(60) Provisional application No. 63/315,286, filed on Mar. 1, 2022.

(51) Int. Cl. A61F 2/24

(52) U.S. Cl. CPC A61F 2/2418; A61F 2/2415; A61F 2210/0009; A61F 2210/0014; A61F 2210/0071; A61F 2220/0008; A61F 2220/0025; A61F 2240/001; A61F 2250/0036

(57) ABSTRACT

Polymeric heart valve systems having a supportive frame embedded in a polymeric sleeve and a plurality of polymeric leaflets forming a continuum with the polymeric sleeve are described herein. The polymeric leaflets are designed to possess variable thickness throughout and typically are formed having a semi-open profile to reduce stress. Methods of making and using such polymeric heart valve systems are also described.

### CROSS-REFERENCE TO RELATED APPLICATION

This application claims benefit and priority to U.S. Application No. 63/315,286, filed Mar. 1, 2022, the disclosure of which is incorporated herein by reference.

### STATEMENT REGARDING FEDERALLY SPONSORED RESEARCH OR DEVELOPMENT

This invention was made with Government Support under Grant Nos. U01 EB026414, and R42 HL134418 awarded by National Institutes of Health. The government has certain rights in the invention.

### FIELD OF THE INVENTION

The invention is generally in the field polymeric heart valve systems and methods of making and using thereof.

### BACKGROUND OF THE INVENTION

Valvular heart disease accounts for significant morbidity and mortality in the U.S. and worldwide. Heart valves function to allow blood movement through heart chambers in an orderly and directionally oriented, forward moving fashion. While durable, native heart valves are subject to a wide range of pathologies which alter function. Two broad categories of valve dysfunction can occur including valvular narrowing leading to valve stenosis, e.g. aortic stenosis; or progressive valvular incompetence leading to valvular regurgitation, e.g. aortic insufficiency. For instance, calcific aortic valve disease (CAVD) is characterized by the development and growth of semi-rigid calcific deposits within aortic valve tissue, where mass buildup results in impaired leaflet motion and an overall increased stiffness of valvular tissue which induces an additional burden on the left ventricle. Such a burden, termed aortic stenosis (AS), is presented as an increased pressure gradient to achieve physiological aortic pressures and cardiac outputs, with progression ultimately leading to reduced flow and related cardiac abnormalities. Thus, clinically valvular heart disease is managed in early and mild stages via medical pharmacologic strategies. However, as valve dysfunction worsens, surgical valve replacement and ultimately valve replacement is required.

Traditionally, valve replacement has been a surgical, open heart surgical therapy. As such, a range of prosthetic or replacement valves have been developed. In recent years, however, advances have been made in obviating the need for surgery for high-risk patients as a means of valve implantation. Percutaneous heart valve implantation has radically changed the approach to treating advanced heart valve disease. As relates to the aortic valve in particular, percutaneous transcatheter aortic valve replacement (TAVR), also termed TAVI (transcatheter aortic valve insertion) has changed our therapeutic approach to advanced aortic valve stenosis. In TAVR, a stent-like scaffold or armature is utilized upon which a bioprosthetic porcine, ovine, or bovine valve is sewed/sutured to the structure, then crimped to low profile and deployed percutaneously or via minimally invasive surgical means (e.g., transfemoral and transapical)

Such transcatheter valves are rapidly becoming the standard-of-care therapy for all aortic valve replacements, however, the currently used tissue-based valve replacement devices, which require suturing during the manufacturing process, are subject to various issues including: damage due to crimping, difficulty in deployment, intrinsic thrombogenicity, limited life span of tissue-based leaflets, high stress off/on leaflets, paravalvular leak risks, lack of accommodation to complex underlying geometry, malpositioning, incomplete expansion, limited or lack of retrievability, infection risks, and structural valve degeneration that can limit their durability and hamper their expanded use in younger lower-risk patients.

Accordingly, there remains a need for valve replacement devices that can address the issues mentioned above.

Therefore, it is the object of the present invention to provide valve replacement devices with improved designs and performance characteristics, such as enhanced durability.

It is a further object of the present invention to provide methods of making such valve replacement devices.

It is still a further object of the present invention to provide valve replacement devices for use in aortic valve replacements in patients in need thereof.

### SUMMARY OF THE INVENTION

Polymeric heart valve systems and methods of making and using thereof are described herein. Current tissue valve manufacturing requires manual suturing of the biologic valve tissue onto a stent which causes the possibility of damage to the tissue and where the suturing holes generate stress concentrations that adversely affect the structural integrity and durability of the valve and may also generate loci for thrombus formation. In contrast, the polymeric heart valve systems described are use suture-less over molding approach to create polymeric leaflets and a polymeric sleeve as a single continuous component, which reduces the risk of calcifications and thrombus formation, biofouling, and infection potential at focal points that may occur in heart valve systems that contain sutures. The design of the polymeric heart valve systems described below achieve variable radial force over the length of the system with higher force aiding in anchoring against the aortic annulus and calcific leaflets while lowering the radial forces on the left ventricular outflow track (LVOT), thus reducing cardiac conduction abnormality (CCA) risk.

The polymeric heart valve system has a supportive frame component. In some instances, the supportive frame is formed of an armature or scaffold-like construct including a plurality of openings and formed from a plurality of connected struts or wire-like elements, wherein each strut or wire-like element is connected to one or more other struts or wire-like elements via a joint. In some other instances, the armature or scaffold-like construct may be in the form of a cage having tubular or wire-like elements configured in a stent-like configuration. In some instances, the armature or scaffold-like construct is formed having a compressed circumferential/axial profile or can otherwise be formed and subsequently crimped, compacted, or collapsed from its size, as formed, into a smaller circumferential/axial profile suitable for implantation, such as through a delivery catheter. The armature or scaffold-like construct is expandable from its crimped, compacted, or collapsed back up to its size, as formed, or any desired size in between. In some instances, the supportive frame is made of a metal or metal alloy which is known to be suitable for medical implantation. In some instances, at least some or all of the surfaces of components (i.e., struts) forming the supportive frame include surface texture features, such as micro and/or macro asperities, which impart surface roughness thereon. The supportive frame may include optional components including one or more anchors, one or more indentations, and/or one or more openings, and these are preferably not covered/coated by any polymer or polymeric matrix.

The polymeric heart valve system has a polymeric sleeve component which enmeshes and encases all or at least a large portion (i.e., greater than 90% of the surface) of the supportive frame. The polymeric sleeve can be formed of any suitable biocompatible, hemocompatible, and mechanically-stable polymer.

In some instances, the polymeric sleeve may include or be marked with one or more radiopaque materials. In some instances, the outer surface of the polymeric sleeve may optionally be at least partially or completely covered with a conformal gel, i.e. what has been termed ectoluminal gel paving, which can seal gaps that may form around the outer sleeve of the polymeric heart valve system, post deployment into a valve, and can serve to minimize the risk of PVL.

The polymeric heart valve system has multiple polymeric leaflets which form a valve component. The polymeric leaflets are connected directly to the polymeric sleeve and are not separately attached to the polymeric sleeve, such as by suturing or other attachment means, where the leaflets and the sleeve are continuous and do not ever form discrete components from one another. The plurality of polymeric leaflets, which can be formed by molding, can be made of the same or a different polymer from which the polymeric sleeve is formed. In some instances, the polymeric leaflets may include or be marked with one or more radiopaque materials. The polymeric leaflets forming the valve can have any suitable dimension, shape, or size needed for purposes of replacing a valve in a subject. In some instances, the polymeric leaflets have variable thickness which can achieve lower stresses within the polymeric leaflets and reduce peak stresses at critical cycle time points during operation of the valve. Thus, the polymeric leaflets each independently have preferably non-uniform thicknesses throughout. Such variable thickness profiles of the leaflets can lead to improved hemodynamics, flexibility, and enhanced or high durability of the polymeric valve systems described. It is noted that this is in contrast to all tissue-based valves, which are used in current valve replacements and have a roughly uniform thickness because these tissues are harvested from animals and cannot incorporate variable thickness therein.

The polymeric heart valve system optionally includes one or more anti-leak flap components. The one or more anti-leak flaps, polymeric leaflets, and polymeric sleeve can be formed of the same polymer/polymeric matrix. The one or more anti-leak flaps can have any suitable dimension, shape, or size needed. The anti-leak flaps are believed to have the ability to block paravalvular leak (PVL) channels by either covering the entrance to the channel, as it covers or seals gaps between the valve system and the native calcific leaflets or by filling the gaps between the valve system and the native calcific leaflets.

Methods of making the polymeric heart valve systems described above are also detailed herein. For example, in one non-limiting instance, a method of making can include the steps of:

(i) placing a supportive frame inside a cavity of a mold in a position suitable to facilitate flow of the polymer around the supportive frame,

wherein the mold comprises a top core, a bottom core, and one or more encasement components which encase the top and bottom cores,

wherein the top core and bottom core define a shape to form polymeric leaflets in a suitable orientation where polymeric leaflets are formed preferably in a semi-open position in a zero residual stress conformation,

optionally, wherein the shape forming the polymeric leaflets has a curvilinear profile along a circumferential axis of the polymeric leaflets being formed; and

(ii) introducing at least one polymer into the mold;

(iii) molding or casting the polymeric leaflets and a polymeric sleeve around the supportive frame to form a polymeric heart valve system,

wherein the polymeric leaflets and the polymeric sleeve are a continuum and each of the polymeric leaflets are connected to the polymeric sleeve at an attachment end; and

(iv) removing the polymeric heart valve system formed from the mold.

The methods described allow for formation of polymeric heart valves where the polymeric leaflets formed avoid punctures because no suturing is needed because the polymeric leaflets and the polymeric sleeve are fully attached to each other.

The polymeric heart valve systems described herein can be used to replace organ valves in a subject, such as human. In some instances, the polymeric heart valve systems can replace defective aortic heart valves. Alternatively, the polymeric heart valve system may be utilized to replace other heart valves; or serve as an extracardiac valve, e.g. in the aorta or other arteries. In still other instances, the polymeric heart valve system is not limited to use only in the heart or structures therein and may be used in veins; or other luminal structures organs, or organ components of the body of a subject.

In some instances, the polymeric heart valve systems can be implanted in a subject in need thereof to replace a defective valve using a non-limiting exemplary method which includes the steps of:

(1) inserting a polymeric heart valve system in a crimped, collapsed, or compacted state into the subject in need thereof through an artery;

(2) delivering the polymeric heart valve system to a defective valve in the subject in need thereof;

(3) implanting the polymeric heart valve system by expanding the crimped, collapsed, or compacted polymeric heart valve system into an expanded state which localizes or fixes the polymeric heart valve system at the defective valve and replaces the function of the defective valve.

In some other instances, the polymeric valve system may be used in a method of treating a subject in need thereof. One exemplary method includes the steps of:

(1′) inserting a polymeric valve system of any one of claims 1-16 in a crimped, collapsed, or compacted state into the subject in need thereof;

(2′) delivering the polymeric valve system to an artery, vein, or luminal structure organ in the subject in need thereof;

(3′) implanting the polymeric valve system by expanding the crimped, collapsed, or compacted polymeric heart valve system into an expanded state, which localizes or fixes the polymeric heart valve system at the artery, vein, or luminal structure organ.

### BRIEF DESCRIPTION OF THE DRAWINGS

FIG. 1A shows a non-limiting illustration of a polymeric heart valve system 100 having a supportive frame 110 embedded in a polymeric matrix forming a polymeric sleeve 120, three polymeric leaflets 130 each being a continuum of the polymeric sleeve without sutures where each of the polymeric leaflets is connected to the polymeric sleeve at an attachment end, an anti-leak flap 135, three crown supporting struts 140, three axial leaflet locking struts 150, and a plurality of optional anchors 160.

FIG. 1B shows a non-limiting illustration of a polymeric heart valve system 100 having three levels: a crown level 170, a calcific leaflet level 180, and a left ventricular outflow track (LVOT) level 190.

FIG. 1C shows a non-limiting illustration of a polymeric heart valve system where an anti-leak flap 135 is located adjacent to the sinus 200, native leaflets 210, and left ventricular outflow track (LVOT) 220 of a heart valve.

FIG. 1D shows a non-limiting illustration of a top view of the polymeric heart valve system with supportive frame 110, polymeric sleeve 120, polymeric leaflets 130, and anti-leak flap 135.

FIG. 1E shows a non-limiting illustration of a top view of the polymeric heart valve system where the anti-leak flap 135 includes a plurality of optional slits 138.

FIG. 2A shows a non-limiting illustration of a top view of three polymeric leaflets 130 of the polymeric heart valve system where each of the polymeric leaflets has variable thickness along the length of the polymeric leaflet.

FIG. 2B shows a non-limiting illustration of thickness variability across an exemplary polymeric leaflet.

FIG. 3A shows a non-limiting illustration of indentation(s) on the edge 310 or in the middle 320 of portions of the struts of the supportive frame.

FIG. 3B shows a non-limiting illustration of an indentation 330 on a joint of the supportive frame 110.

FIG. 4A shows a non-limiting illustration of a partial section of the polymeric heart valve system with supportive frame 110, polymeric sleeve 120, a polymeric leaflet 130, and anti-leak flap 135 against a native leaflet of the heart valve.

FIG. 4B shows a non-limiting illustration of a partial section of the polymeric heart valve system where the anti-leak flap 135 is placed in partial supra-annular position against a native leaflet of the heart valve.

FIG. 4C shows a non-limiting illustration of a partial section of the polymeric heart valve system where the anti-leak flap 135 is placed in supra-annular position against a native leaflet of the heart.

FIG. 5A shows a non-limiting representation of a mold 300 having a base/ventricular core 310, encasement components 320, a top/aortic core 330 having multiple axial leaflet lock registrations 340 which are placed around a supportive frame 110.

FIG. 5B shows a non-limiting representation of a base/ventricular core 310, encasement component 320, a top/aortic core 330 having multiple axial leaflet lock registrations 340 after formation of a polymeric heart have system 100 therein.

FIG. 5C shows a non-limiting cross-sectional representation of a closed mold having a base/ventricular core 310, a top/aortic core 330, and encasement component 320 where a polymer is introduced by pressure using a plunger 350 and the polymer flows throughout the mold (white arrows).

FIG. 5D shows a non-limiting cross-sectional representation of a portion of a mold around where a polymer 360 is flowing around a supportive frame 110 to form a polymeric sleeve thereon.

FIG. 5E shows a non-limiting representation of a base/ventricular core 310 and a top/aortic core 330 where the core components are designed to produce polymeric leaflets having a closed position.

FIG. 5F shows a non-limiting representation of a base/ventricular core 310 and a top/aortic core 330 where the core components are designed to produce polymeric leaflets having a semi-open position.

FIGS. 6A and 6B are graphs of the maximum absolute principal stress within three 27 mm leaflet profiles over the cardiac cycle pressure waveform, with the top graph (6A) demonstrating the tensile stresses and the bottom graph (6B) the compressive stresses. The more open profiles have lower systolic stresses and higher diastolic stresses. The more closed profile has lower stresses overall.

FIG. 7 is an illustration and graph showing the validation of the polymeric leaflets motion with the comparison of geometric orifice area (GOA) and the FEA profile. The line graph compares the GOA of each model throughout systole.

### DETAILED DESCRIPTION OF THE INVENTION

Polymeric heart valve systems and methods of making and using thereof are described below.

### I. Definitions

The term “biocompatible,” as used herein, refers to a material, such as a polymer, which performs its desired function when introduced into an organism without inducing significant inflammatory response, immunogenicity, or cytotoxicity to native cells, tissues, or organs.

The term “hemocompatible” refers to a set of properties of a material that allow for contact with flowing blood without causing adverse reactions such as thrombosis, hemolysis, complement activation, bleeding, or inflammation.

The term “mechanically stable” refers to the ability of a material to maintain its integrity over time when subjected to one or more external stresses.

The terms “conformal,” or “conformally coated,” as used herein typically refer to covering a surface topography of an object with a material such that it is completely or effectively covered up to at least about 50%, 60%, 70%, 80%, 90%, 95%, 96%, 97%, 98%, or 99% of the surface area which is intended to be covered by the material without exposure of the underlying material of the object where covered. The conformal coating is considered to be in direct contact, in intimate contiguity, and matches the geometry and contour to the surface the coating is applied to, so as to act as a cover, barrier, or shield or otherwise form a barrier layer preventing exposure of the underlying surface to exogenous contact by at least about 50 to 100%, as compared with no conformal coating.

The term “subject” refers to either a human or non-human animal.

The term “treating” refers to inhibiting, ameliorating, impeding, alleviating, or relieving a disease, disorder, or condition from occurring in a subject or causing regression of the disease, disorder, and/or condition. Treating the disease or condition includes ameliorating at least one symptom of the particular disease or condition, even if the underlying pathophysiology is not affected.

Numerical ranges disclosed in the present application include, but are not limited to, ranges of temperatures, ranges of pressures, ranges of integers, ranges of times, and ranges of thicknesses, etc. The disclosed ranges of any type, disclose individually each possible number that such a range could reasonably encompass, as well as any sub-ranges and combinations of sub-ranges encompassed therein. For example, disclosure of a thickness range is intended to disclose individually every possible thickness value that such a range could encompass, consistent with the disclosure herein.

Use of the term “about” is intended to describe values either above or below the stated value, which the term “about” modifies, in a range of approx. +/−10%; in other instances the values may range in value either above or below the stated value in a range of approx. +/−5%. When the term “about” is used before a range of numbers (i.e., about 1-5) or before a series of numbers (i.e., about 1, 2, 3, 4, etc.) it is intended to modify both ends of the range of numbers or each of the numbers in the series, unless specified otherwise.

### II. Polymeric Heart Valve Systems

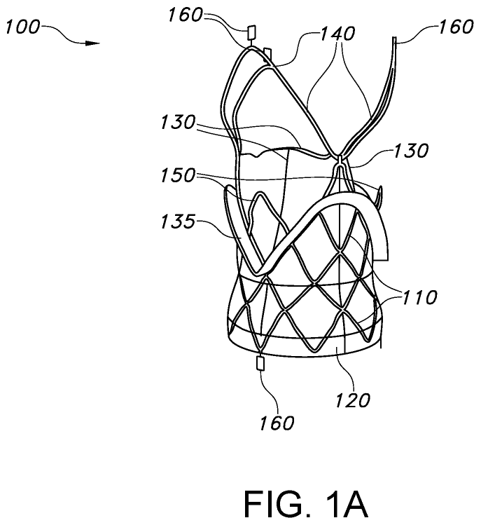

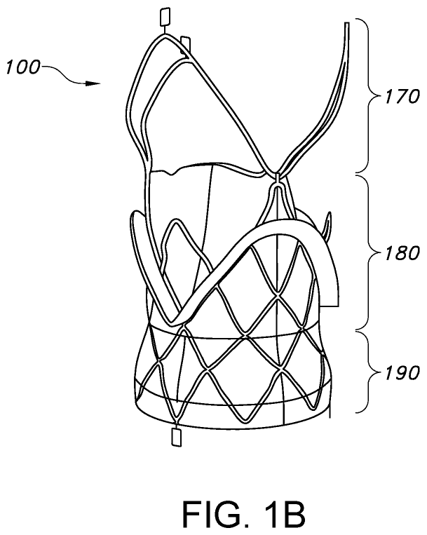

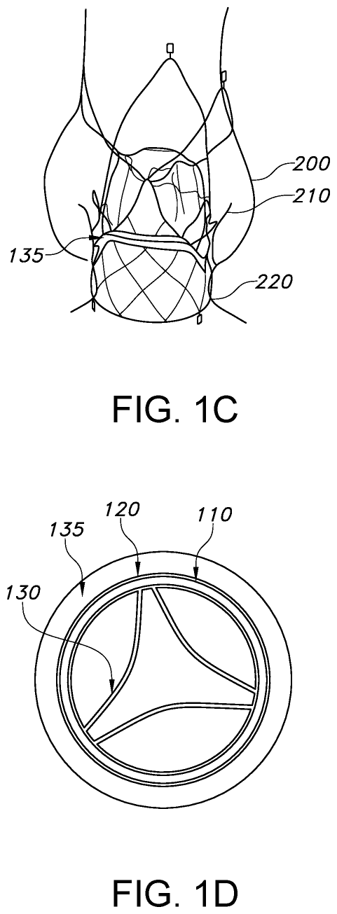

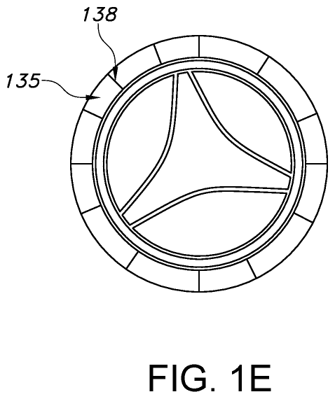

Polymeric heart valve systems are described herein. As shown in FIG. 1A, a polymeric heart valve system 100 includes a supportive frame 110 embedded in a polymeric matrix forming polymeric sleeve 120, a plurality of polymeric leaflets 130 each being a continuum of the polymeric sleeve without sutures where each of the polymeric leaflets is connected to the polymeric sleeve at an attachment end, an anti-leak flap 135, a plurality of crown supporting struts 140, a plurality of axial leaflet locking struts 150, and a plurality of optional anchors 160. Moreover, as shown in FIG. 1B, the polymeric heart valve system 100 having three levels: a crown level 170, a calcific leaflet level 180, and a left ventricular outflow track (LVOT) level 190. The crown supporting struts and axial leaflet locking struts are preferably not embedded in the polymeric matrix forming the polymeric sleeve. Further, the plurality of leaflets are able to open and close at an operative end, wherein when the plurality of leaflets are in the closed position, the operative ends of the leaflets abut each other.

As described in detail further below, the polymeric heart valve system can be formed by placing a supportive frame in a mold and a suitable polymer can be used to form or cast to the plurality of leaflets of a given design and polymeric sleeve which around or embed a supportive frame which can be, for example, an expandable stent.

Current tissue valve manufacturing requires manual suturing of the biologic valve tissue onto a stent which causes the possibility of damage to the tissue and where the suturing holes generate stress concentrations that adversely affect the structural integrity and durability of the valve and may also generate loci for thrombus formation. Without wishing to be bound by any particular theory a suture-less over molding approach to create the leaflets and polymeric sleeve of the heart valve systems described, as part of a single manufacturing step, reduces the risk of calcifications and thrombus formation, biofouling, and infection potential at focal points that may occur in heart valve systems that contain sutures. Moreover, continuous wrapping of the polymeric sleeve around the supportive frame and the adhesion of the polymer to the supportive frame allows even distribution of the leaflet forces and stresses during operation. Polymeric components of the system can address or overcome known clinical complications of persistent thrombosis events, such as hypo-attenuated leaflet thickening (HALT) and stroke, as well as the limited durability of tissue-based (i.e., bovine/ovine/porcine) heart valve systems. Polymeric materials can also offer reduced platelet response and adhesion with smoother, less-fibrous surfaces and hemocompatible surface chemistries.

Additionally, the design of the polymeric heart valve systems described herein achieves variable radial force over the length of the system with higher force aiding in anchoring against the aortic annulus and calcific leaflets while lowering the radial forces on the left ventricular outflow track (LVOT), thus reducing cardiac conduction abnormality (CCA) risk. The leaflet shape, combined with varying leaflet thickness, leads to reduction in the flexural cyclic stresses and the polymeric heart valve's hydrodynamics. The crown level or region of the polymeric heart valve system contacts the sinotubular junction of the aorta and should produce lower radial forces to avoid damage to the aorta. In some instances, this can be achieved with three longer contacting joints incorporated into the system. Lower radial force in the LVOT section can be achieved with longer crown supporting struts and additional radial supporting struts which aid in sealing against intra and/or paravalvular leaks.

PVL channels are complex and highly restricted flow paths due to incomplete sealing between an expanded TAVR device and underlying calcified leaflets and the aortic wall that are driven by large diastolic pressure gradients, creating high velocity jet flows from the native sinuses back into the left ventricular outflow track (LVOT). PVL is often classified by leak severity determined by clinician judgement of the jet velocity and flow.

### A. Components of Polymeric Heart Valve System

The various components of the polymeric heart valve system are described in detail below.

### 1. Supportive Frame

In some instances, the supportive frame is formed of an armature or scaffold-like construct including a plurality of openings and formed from a plurality of connected struts or wire-like elements, wherein each strut or wire-like element is connected to one or more other struts or wire-like elements via a joint. In some other instances, the armature or scaffold-like construct may be in the form of a cage having tubular or wire-like elements configured in a stent-like configuration. In some instances, the armature or scaffold-like construct is formed having a compressed circumferential/axial profile or can otherwise be formed and subsequently crimped, compacted, or collapsed from its size, as formed, into a smaller circumferential/axial profile suitable for implantation, such as through a delivery catheter. The armature or scaffold-like construct is expandable from its crimped, compacted, or collapsed back up to its size, as formed, or any desired size in between. Preferably, the armature or scaffold-like construct is an expandable stent which is self-expandable or otherwise requires active expansion by balloon expansion or heat expansion. Expandable stents are plastically deformed with mechanical energy, such as by balloon expansion, into an “opened” or deployed shape. Self-expandable stents can be thermally fixed or shape-set after expansion. Such expandable stents and materials for making such stents are known in the art.

In some instances, the total diameter of the supportive frame embedded in the polymeric sleeve, when crimped, compacted, or collapsed is about 4, 5, 6, or 7 mm and can be delivered through an artery, such as the femoral artery. The polymeric sleeve embedded supportive frame once crimped, compacted, or collapsed can be expanded or returned back to its original size or to a total diameter size ranging from about 15 to 30 mm, 20 to 30 mm, 25 to 30 mm, or to a total diameter size of about 25, 26, 27, 28, 29, or 30 mm in (re) expanded form. In some instances, it is better to avoid crimping of the as-formed polymeric heart valve system until it is time to load it into a delivery catheter, such that the first crimping, compacting, or collapsing of the polymeric heart valve system is during loading into a catheter, or placement or affixing onto a delivery deployment device.

In some cases, once the polymeric heart valve system is inserted/deployed into a body valve and fully expanded so there is oversizing, which refers to how much the expanded supportive frame of the polymeric heart valve system is constricted by the surrounding anatomy (i.e., diameter of valve orifice). Oversizing can be used to produce a radial force in order to keep the polymeric heart valve system in a desired position in situ, such as by frictional fit or similar mechanical locking in place. Such constriction is usually proportional to how much radial force the expanded supportive frame of the polymeric heart valve system can exert on the anatomy (e.g., valve orifice in the heart) to keep the system in place and avoid migration of the polymeric heart valve system. For the supportive frame, oversizing of about 0 to 40%, about 0 to 30%, about 0 to 20%, about 0 to 15%, about 0 to 10%, or about 0 to 5% and sub-ranges within are acceptable. In some instances, oversizing can be dependent on the radial force that the supportive frame of the system can exert. For example, oversizing of the supportive frame in a relaxed state is considered to be 0%. Oversizing of the supportive frame in the mold can range from about 0 to 8%. Finally, oversizing of the supportive frame, as part of the system, when used in vivo can range from about 5 to 50%, about 5 to 40%, about 5 to 30%, about 5 to 20%, about 5to 10%, or about 10 to 15%, as well as sub-ranges within. The extent of oversizing can also depend on the anatomical location of implantation of the polymeric heart valve system.

In some instances, the supportive frame is made of a metal or metal alloy which is known to be suitable for medical implantation. Without limitation, the supportive frame can be formed of self-expanding memory metal alloys, such as NiTiCo, NiTiCr, NiTiCu, NiTiNb, or NiTi (Nitinol) or other known expandable metal/metal alloys, such as spring steels, stainless steel, platinum, tantalum alloys, or cobalt chromium. If actively expandable, the supportive frame can be made of biocompatible metals of particular types, including stainless steel (316L, 304L), cobalt-chromium alloys (L605), nickel-titanium alloy (Nitinol), platinum, and tantalum alloys.

In some instances, the struts or wire-like elements forming the supportive frame can have any suitable dimensions (i.e., length, thickness, radial diameter, etc.). In some cases, the struts or wire-like elements have a tubular shape with a uniform radial tube diameter and the radial thickness ranges from between about 0.2 to 0.6 mm. In some instances, the radial thickness is about 0.3 mm. In some other cases, the struts or wire-like elements are not tubular but have a uniform thickness throughout the struts or wire-like elements and the thickness ranges from between about 0.2 to 0.6 mm. In some other instances, the strut or wire-like elements are curvilinear and can have a variable circumferential thickness along the length of the strut or wire-like element where the thickness of the strut or wire-like element has a 150-250 μm circumferential size and a 250-350 μm radial size, and any sub-ranges or individual values disclosed therein.

In some instances, the struts or wire-like elements of the supportive frame, regardless of shape, are (electro) polished which reduces their thickness prior to the supportive frame being embedded in the polymeric sleeve. Further, (electro) polishing can be performed to remove sharp features that would add stress concentrations, to aid in reducing crimping, compacting, or collapsing strain and resisting plastic deformation during crimping, compacting, or collapsing, as well as increasing the fatigue life/resistance of the formed supportive frame. Additionally, polishing can prepare blood contacting surfaces for better hemocompatibility or provides a more favorable surface morphology to resist thrombus development. In some instances, prior to or following (electro) polishing, the supportive frame may be subjected to sand blasting or chemical etching or other surface modifying method to generate texture and asperities, to the frame surface which can aid in adhesion of the polymer matrix during coating and formation of the polymeric sleeve on the frame; and/or aid in enhancing the durability of adhesion to the frame over use life. In some cases, the supportive frame can be masked before chemical etching or sand blasting to ensure only (electro) polished surface(s) are exposed to blood flow.

The supportive frame may be formed by any suitable method known in the art. Methods of fabrication can include, but are not limited to, laser cutting or etching, laser forming, wire braiding or bending, metal etching, metal vapor deposition, 3-D metal printing, precision machining/CNC, chemical etching, spray coating, sputter coating, powder coating, additive manufacturing, and other art known techniques used to form a stent-like armature or scaffold-like construct suitable for implantation in a subject. As an example, in some instances, a stock material maybe extruded into a cylinder or generated by art known vapor deposition methods for controlled alloy/composite structuring. The cylinder can be, for example, cut with a laser or lathe into a desired shape having a compressed 2D circumferential/axial profile. The compressed profile and stock tube size dictates the minimum compressed size of the supportive frame. In such non-limiting instances, the initial supportive frame can be shape set with expanding or step-wise increasing with a mandrel or jig. The shape setting process can include a single or multiple steps to avoid damaging the structural integrity of the supportive frame. In one non-limiting instance, the supportive frame is made by laser cutting from nitinol tubes and then performing thermal shape-setting that includes expansion of the supportive frame using dedicated jig(s) and temporal exposure to elevated temperature (e.g., about 450° C. to 550°° C., or, in some instances, about 520° C.). In forming such supportive frames, shape-setting can be performed in single step or in multiple steps (where in each step the supportive frame is incrementally expanded to the desired size) and full or partial electropolishing may optionally be performed during these shape-setting steps. Sandblasting and/or electropolishing may be performed after all shape-setting steps are completed. Other methods of making implantable stents are described in U.S. Pat. No. 8,715,335; 10,729,824; 10,806614; 10,874,532; 11,045,297; 11,058,564. In less preferred instances, the supportive frame may be formed of polymer(s), such bio-degradable polymers known in the art.

In some instances, at least some or all of the surfaces of components (i.e., struts) forming the supportive frame include surface texture features, such as micro and/or macro asperities, which impart surface roughness thereon. The surfaces may have morphologies ranging from fully smooth and featureless (e.g. on once face—i.e. adluminal) to others having micro and/or macro asperities (e.g. abluminal face). However, such surface morphologies having asperities are found only on one or more surfaces that are not exposed, for example, to blood flow in order to prevent risk of thrombus formation. Such surface roughness may be imparted by various methods including, but not limited to, plasma etching or chemically etching the supportive frame prior to embedding the frame in a polymeric matrix. Methods and conditions for plasma and chemical etching are known to those skilled in the art. In some other instances, the surfaces of components (i.e., struts) forming the supportive frame can have a suitable primer coating that can chemically bind polymer(s) applied thereto. In one non-limiting example, one or more primer coatings may include a first coat of identical or similar polymer as in the final polymeric sleeve, or other polymers providing adhesive properties, increased durability properties, or additional functionalities, such as providing device radiographic opacity. In some instances, a primer coating may be made of or include silicone polymers.

The supportive frame and struts or wire-like elements can be designed to selectively produce a radial force within a portion or segment of the polymeric heart valve system (such as at the calcific leaflet level; see FIG. 1B) or a lowest or minimal crimping, compacting, or collapsing strain, while maintaining the lowest surface area or volume, when embedded in the polymeric sleeve.

As noted above, in some instances, the supportive frame and the strut or wire-like elements from which it is formed are curvilinear where the curved shape allows for bending motion during crimping, compacting, or collapsing occur over the length of the strut or wire-like element and is not constrained to a joint region between the strut or wire-like elements. Without wishing to be bound by any particular theory the larger bending curvature can allow more polymeric matrix material to be cast during molding to embed the joint regions without risking increased shearing stresses against the polymeric matrix material that may lead to damage during crimping, compacting, or collapsing of the supportive frame.

a. Optional Supportive Frame Components

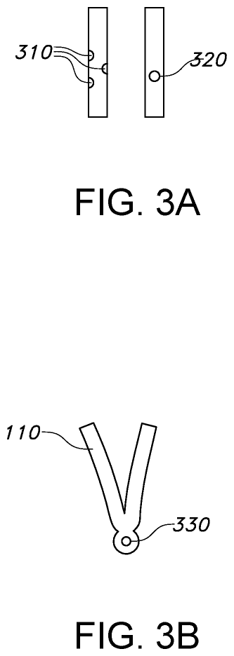

The supportive frame may include optional components including one or more anchors, one or more indentations, and/or one or more openings, and these are preferably not covered/coated by any polymer or polymeric matrix. See FIG. 1A for exemplary non-limiting examples of anchors (160) and FIGS. 3A and 3B for exemplary non-limiting examples of indentations which may form part of the strut or wire-like elements of the supportive frame.

In some instances, one or more anchors which may form part of the strut or wire-like elements of the supportive frame are located on extreme ends of struts the supportive frame. For example, anchors may be placed on the crown supporting struts (140) or wire-like elements of the crown level and/or supporting frame at the LVOT level of the frame. The anchors form part of the supportive frame and can be formed during the manufacture of the frame.

The anchors may have any suitable size and shape. In some instances, the anchors form square, rectangular, circular, or oval ring shapes. The anchors, in some instances, are designed to interface the supportive frame into a mold and hold it and constrain the supportive frame during the molding process to embed it in the polymeric sleeve. The anchors, in some other instances, can serve as imaging marks during deployment of the polymeric heart valve system when the anchors are marked with a radiopaque material or metal. Exemplary imaging marker materials can include, but are not limited to, gold, tantalum, or platinum-iridium. It is possible to mechanically connect a piece of the imaging marker material to specific regions in the supportive frame, which can be designed specifically for that purpose (e.g. leaving a void for placing the marker). In such instances, when used as imaging marks, the anchors designate the top and bottom of the polymeric heart valve system and can be used to find the location of leaflets.

In some instances, one or more indentations and/or one or more openings may form part of the strut or wire-like elements of the supportive frame, as shown in FIGS. 3A and 3B. The indentations can be used for mechanically locking the polymeric sleeve to the supportive frame. Such indentation or openings can be spread along the supportive frame so as to minimize separation and sliding of the polymer on the struts or wire-like elements of the supportive frame during crimping/compacting/collapsing of the embedded supportive frame, as well as during deployment (i.e., implantation in a subject) and expanding the supportive frame of the system to localize/implant it in a valve. The indentations or openings can be placed in the middle of the struts or wire-like elements of the supportive frame, a region where deformation of the strut is believed to be relatively small, such that the indentation would not impair the mechanical stability of the stent. The indentations or openings can be formed during or following the manufacture of the supportive frame and prior to embedding the frame in the polymeric sleeve. In some instances, the indentations can have a depth of about 10 to 100 μm or a depth of about 10, 20, 30, 40, 50, 60, 70, 80, 90, or 100 μm, in relation to a non-indented portion of the struts or wire-like elements.

### 2. Polymeric Sleeve

As described above, the supportive frame is embedded in a polymeric matrix that forms a polymeric sleeve which enmeshes and encases all or at least a large portion (i.e., greater than 90% of the surface) of the supportive frame at least at the calcific leaflet and LVOT levels. See FIG. 1B. In some instances, portions of the supportive frame, such as the axial leaflet locking struts (150) at the calcific leaflet level (180), are not embedded or otherwise coated with any polymeric matrix or sleeve.

In some instances, the thickness of the embedding polymeric matrix forming the polymeric sleeve can have any suitable thickness thereon. The thickness of the extraluminal polymeric matrix material covering the supportive frame can, for example, when formed by compression molding or other molding techniques have a thickness in a range from about 0 to 500 microns in the circumferential direction and/or a thickness ranging from about 0 to 400 microns in the radial direction of the struts or wire-like elements of the supportive frame. In some instances, an average thickness of about 400 microns in the circumferential direction (i.e. about 200 microns are on each side of the strut) is present. A thickness value of zero, as noted above, can occur in certain cases where the supportive frame is in contact with a mold surface during the embedding of the frame in the polymeric supportive sleeve and some regions of exposed supportive frame could have no or about zero thickness of polymeric matrix thereon. In other instances, where the supportive frame is embedded in a polymeric matrix material by, for example, dip coating the frame into polymer solution, the encapsulation thickness of the struts or wire-like elements of the supportive frame is expected to be smaller with a thickness ranging from about 0 to 200 microns in the circumferential and/or radial directions.

The polymeric sleeve can be formed of any suitable biocompatible, hemocompatible, and mechanically-stable polymer. In preferred instances, the polymeric material will have elastomeric properties allowing for expansion and reconfiguration following deployment, once situated in its deployment location. The polymeric sleeve should not separate from the supportive frame in use and over time. Such suitable polymers are art known for use in medical implants, and possible blends of such polymers are also contemplated. In some instances, the polymers used to form the polymeric sleeve are materials which can also offer reduced platelet response and adhesion with smoother, less-fibrous surfaces and hemocompatible surface chemistries.

In some instances, the surface(s) on the outer face of the polymeric sleeve can be made rougher than the surface of the inner surface of the polymeric sleeve (valve lumen) in order to allow for better adhesion and integration of the outer, i.e. ectoluminal surface, to the native tissue in which the valve system is being implanted on.

Without particular limitation, exemplary polymers which can be used to form the polymeric sleeve during molding include thermoplastic polymers/elastomers, thermoset polymers, thermally crosslinkable polymers, elastomeric polymer biomaterials, polymers containing polysulfone(s), polyurethanes, silicones, PTFE, crosslinked poly (styrene-isobutylenese-styrene) (xSIBS), and poly (styrene-isobutylene-styrene) (SIBS). Thermoplastic elastomers (TPEs) can be formed of or contain polymyrcene, polymenthide, and poly (ϵ-decalactone). Elastomeric biomaterials can include, for example, silicones, thermoplastic elastomers, polyolefins, polydiene elastomers, poly (vinyl chloride), natural rubbers, heparinized polymers, hydrogels, polypeptides elastomers, polysiloxane-urea elastomers, and polyurethanes. As appropriate, the skilled person would be able to select conditions (i.e., temperatures, solvent(s), etc.) needed to form polymer melts or form polymer solutions of the aforementioned to be able to embed the supportive frame during molding, as detailed further below.

In some instances, the polymer material which forms the polymeric sleeve may include one or more fiber reinforcement materials. Such fiber reinforcement materials can be formed of or contain, but are not limited to, poly-alkanes, polyethylene, polytetrafluoroethylene, polyamide, polypropylene, polyethyleneterephthalate, polydimethylsiloxane, polyhydroxyalkanoates, polymethylmethacrylate, silicone, parylene, polydimethylsiloxane, SU-8, liquid crystal polymers, polyurethane, polyetherketones, biodegradable polymers. Exemplary biodegradable polymers can include, without limitation, polylactide (PLA), polyglycolide (PGA), polycaprolactone (PCL), and copolymers or blends thereof.

In some instances, the polymeric sleeve may include or be marked with one or more radiopaque materials. Exemplary radiopaque materials which can be added to the above polymers forming the sleeve can include, but are not limited to, stainless steel, gold, tantalum, or platinum-iridium, barium, iodine, and alloys, blends, and mixtures thereof.

a. Gel Paving

In some instances, the outer surface of the polymeric sleeve may be at least partially or completely covered with a conformal gel, i.e. what has been termed ectoluminal gel paving, which can seal gaps that may form around the outer sleeve of the polymeric heart valve system, post deployment into a valve, and can serve to minimize the risk of PVL.

In some instances, the outer surface of the polymeric sleeve is enveloped with a desiccated hydrogel coating. Post-deployment into a defective valve in a subject, the desiccated gel is subject to rehydration in situ which will result in gradual swelling of the gel which will form a thicker, outer facing, gel layer, which is free to swell filling any irregular gaps between the polymeric heart valve system and valve wall. Endoluminal gel swelling may be limited by: constraint of the gel by the supportive frame, the underlying polymeric sleeve and the minimal swollen thickness of the desiccated hydrogel layer. However, it is believed that sealing and regional efficacy of leak resistance will be increased following by using such conformal gel paving on the polymeric sleeve of the system.

Exemplary gel paving materials can include, but are not limited to, polymeric hydrogels derived from diacrylate poly (ethylene) glycol (PEG) macromers, which can serve as hemocompatible hydrogel networks, along with macromers incorporating oligocarbonate units. Incorporation of oligocarbonate flanks on PEG before acrylation are able to extend gel durability. In some other instances, non-degradable hydrogels alternatives can be employed, which do not contain degradable units installed into the copolymer.

Gel paving hydrogels can be desiccated, with gels shrinking to an extremely low profile (being 90% water) to allow crimping and compaction for valve system deployment. Once deployed, with unsheathing, the hydrogels will gradually re-swell (typically in about 2 to 10 min, with up to a 2 to 9-fold increase in volume), regenerating the hydrogel network to efficiently fill-in irregular-shaped gaps and seal the polymeric heat valve system to the surrounding valve, such as aortic valve, preventing paravalvular leakage.

In some cases, a hydrogel paving material is placed on the polymeric sleeve outer surface by first enveloping a mesh with the material that will be integral to the polymeric sleeve. This enmeshment facilitates durable bonding, adhesion and resistance to peel, despite crimping and re-expansion of the valve system during deployment.

In some instances, dessicated hydrogel is applied on the outer surface of the polymeric sleeve in a range from between about 0.5 to 5 mm. The gel paving material will swell to fill gaps, many of which may be of varying sizes and shapes.

### 3. Polymeric Leaflets

The valve mechanism of the polymeric heart valve system includes a plurality of individual polymeric leaflets, where the polymeric leaflets form a continuum with the polymeric sleeve. See FIG. 1A. In other words, the polymeric leaflets are connected directly to the polymeric sleeve and are not separately attached to the sleeve, such as by suturing or other attachment means, where the leaflets and the sleeve are continuous and do not ever form discrete components from one another.

The suture-less feature of the polymeric leaflets of the system can be achieved, for example, by forming the polymeric leaflets and polymeric sleeve (onto the supportive frame) at the same time during the molding process (i.e., single manufacturing step) whereby they form a single polymeric component containing both the polymeric leaflets and polymeric sleeve. In less preferred instances, polymeric leaflets may be formed/molded as a first component and then the leaflets and a supportive frame are dip coated into a suitable polymer melt or solution to form the polymeric sleeve. During such instances, the polymer melt or solution forms the polymeric sleeve over the supportive frame and simultaneously attaches to the polymeric leaflets in the mold. Thus, the leaflets become a continuous inseparable part of the formed polymeric sleeve with smooth transitions forming a “continuum.”

The plurality of polymeric leaflets which can be formed by molding can be made of the same or a different polymer from which the polymeric sleeve is formed. In most instances, the polymeric leaflets and polymeric sleeve are formed of the same polymer/polymeric matrix. Exemplary polymers which can be used to form the polymeric leaflets include, but are not limited to, thermoplastic polymers/elastomers, thermoset polymers, thermally crosslinkable polymers, elastomeric polymer biomaterials, polymers containing polysulfone(s), polyurethanes, silicones, PTFE, crosslinked poly (styrene-isobutylenese-styrene) (xSIBS), and poly (styrene-isobutylene-styrene) (SIBS). Thermoplastic elastomers (TPEs) can be formed of or contain polymyrcene, polymenthide, and poly (ϵ-decalactone). Elastomeric biomaterials can include, for example, silicones, thermoplastic elastomers, polyolefins, polydiene elastomers, poly (vinyl chloride), natural rubbers, heparinized polymers, hydrogels, polypeptides elastomers, polysiloxane-urea elastomers, and polyurethanes. As appropriate, the skilled person would be able to select conditions (i.e., temperatures, solvent(s), etc.) needed to form polymer melts or form polymer solutions of the aforementioned to be able to form the plurality of leaflets during molding, as detailed further below. In some instances, thermoplastic polymers can be chosen when instances of dip coating are used to form the systems described and thermoset polymers can be chosen when instances of molding are used to form the systems described. In still other instances, a combination of both dip coating of the polymeric sleeve and to attach them to previously molded polymeric leaflets can be used.

In some instances, the polymeric leaflets may include or be marked with one or more radiopaque materials. Exemplary radiopaque materials which can be added to the above polymers forming the polymeric leaflets can include, but are not limited to stainless steel, gold, tantalum, or platinum-iridium, barium, iodine, and alloys, blends, and mixtures thereof.

The polymeric leaflets forming the valve can have any suitable dimension, shape, or size needed for purposes of replacing a valve in a subject. Typically, the design dimensions of the valve and polymeric leaflets therein are based on the free or open deployed diameter of the supportive frame when implanted in a subject. In some cases, the valve height and leaflet height can range from about 0.5 to 2 times the diameter of the free or open deployed diameter of the supportive frame when implanted in a subject. The valve or leaflet height can tailored and selected for individualized sizes based on a subject's valve size.

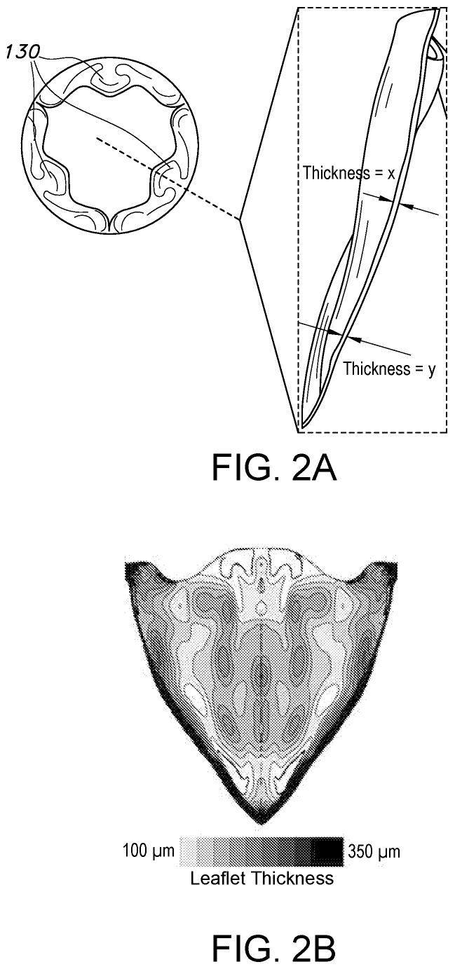

In most instances, the valve system includes three polymeric leaflets (130) which each have a curvilinear or wavy profile in a circumferential axis and are preferably formed/molded in a semi-open conformation. See FIG. 2. Without wishing to be bound by any particular theory, the semi-open profile aids in minimizing both overall stresses (i.e., flexural stresses) in the valve and during formation of the valve in molding. Further, a semi-open profile can lower the stresses in the polymeric leaflets during systole when the valve's leaflets are fully open, as well as lowering the typical increased stresses acting on the closed leaflets during diastole. Nevertheless, other leaflet conformations ranging from fully open to fully closed conformations can also be formed/molded. Selection of a particular configuration (i.e., open, semi-open, closed, and variations therein) can be based on the ease of molding the desired profile and/or to provide a balance between lower systolic and lower diastolic stresses when the valve is in use, preferably to keep the stresses throughout the valve cycle lower and below the fatigue limit of the polymer forming the polymeric sleeves and leaflets.

a. Variable Thickness

Without being bound to any particular theory, the use of variable thickness polymeric leaflets in the valve system can achieve lower stresses within the polymeric leaflets and reduce peak stresses at critical cycle time points during operation of the valve.

The particular pattern of thicknesses within each leaflet can be determined by mapping the time-dependent principal stress distribution of a model of the polymeric leaflets over the duration of a typical cardiac cycle. In a first step, a model thereof is formed and moved through a typical cycle to determine or estimate the locations and relative amounts, e.g. high versus low, of stress in each location throughout a typical cycle for all or one or more regions within the polymeric leaflet. The relative amounts of stress that can be analyzed correspond with the bending, twisting, stretching, or other motions which can occur during a typical diastole and systole cardiac cycle. Based on the mapping of such stresses, the particular locations and relative thicknesses for the leaflets are selected to reduce or minimize stress during cycles of use. Interactive finite element analysis (FEA) can be used to produce/model the aforementioned the time-dependent principal stress distribution. A model of the polymeric leaflets can refer to a physical model formed from a material that is the same as or different from the material of the final leaflets. Optionally, the model is a digital model, such as one created using software, which can be digitally manipulated to determine or estimate the relative stresses and their locations during a typical diastole and systole cardiac cycle.

Thus, the polymeric leaflets each independently have preferably non-uniform thicknesses throughout. In some instances, the variable thickness is understood by considering a center line of symmetry of each of the polymeric leaflets, where in a given cross-section each of the leaflets has at least two or more thicknesses. In some instances, the polymeric leaflets each independently have a non-uniform (i.e., variable) thickness across any cross-sectional direction of the leaflet. In other words, the polymeric leaflets have a variable thickness profile which is not uniform across any of the leaflet's circumferential cross-sections, and without any preferential directionality in the thickness within such cross-sections. Such variable thickness profiles of the leaflets can lead to improved hemodynamics, flexibility, and enhanced or high durability of the polymeric valve. In some instances, enhanced or high durability can be defined based on the FDA ISO 5840 (2013) guidelines which defines that such a valve system, as described herein, would survive at least 200 million cycles or more of rapid valve opening and closing (in a special durability tester)-equivalent to about 5 years operation in a subject (patient). In some instances, the polymeric valve system described can perform up to or at least 1 billion cycles, and potentially more cycles going on (equivalent to about 25 years in a subject). The polymeric heart valve systems described, and methods of use thereof, can provide valve replacements with durability exceeding current standards for valve testing and durability.

In some non-limiting instances, the thickness at one position of a polymeric leaflet will differ from the thickness at another/different position on the same leaflet. The thicknesses across a longitudinal cross-section of the leaflet may vary with a specified slope, such that the thickness decreases or increases in a given direction of the cross-section. For example, as shown in FIG. 2A, thickness “x” at a first position of a cross-section of an exemplary leaflet is greater than the thickness “y” at a second position. FIG. 2B shows a representation of thickness variability present in an exemplary polymeric leaflet. In still other instances, the thickness from any given position on the polymeric leaflets varies as one moves radially out in any direction to another position.

In some instances, the polymeric leaflets, which are connected to the polymeric sleeve at an attachment end, have a thickness which is greatest at the attachment end to the sleeve and which decreases with distance away from the attachment end in any direction taken until a terminal edge of the leaflet is reached. In other words, the polymeric leaflets may have thicknesses that become thinner moving out from the attachment edge. In some instances, a maximum thickness of any portion of the polymeric leaflets can range from between about 200 to 600 μm or about 400 to 500 μm, as well as sub-ranges within. The maximum thickness may be dictated by the volume of the leaflets which should be reduced for ease of crimping/compacting/collapsing the formed valve system and for effective reduction of the overall stresses on the system. In some instances, a minimum thickness of any portion of the polymeric leaflets can range from between about 50 to 200 μm, about 50 to 150 μm, or about 50 to 100 μm, as well as sub-ranges within. In still other instances, any portion of the polymeric leaflets have a variable and non-uniform thickness can have thickness ranging from between about 50 to 600 μm. The desired thicknesses of any part or portion of each of the respective leaflets can be controlled by way of the molding technique and the lowest limit of capable thickness which can be achieved.

Without wishing to be bound by any particular theory a benefit of variable thickness in the polymeric leaflets is in achieving a maximum peak von Mises stress which is at or below about 30%, 35%, 25%, 20%, 15%, or 10% of the yield stress of the polymer it is composed from, as well as to provide lower and more uniform stress distribution during peak diastole and can reduce bending stresses during systole which reduces the average stresses throughout the entire cardiac cycle. Thus, polymeric leaflets with variable thickness can exhibit lower overall stresses while also providing an overall reduction in the volume of material used in forming the leaflets. This can also benefit the ability to crimp/compact/collapse the polymeric heart valve system due to the decreased volume. Reduction in the polymer volume within the polymeric leaflets can help reduce the size of the valve system for easing delivery of the valve to a subject. In some instances, use of variable thickness polymeric leaflets includes a reduction in peak principal stress (absolute) at or below about +1.2 MPa and above about −1.0 MPa and a reduction in von Mises stresses at below about 1 MPa, with a lower, more uniform stress distribution during peak diastole.

The variable thickness in the polymeric leaflets can be achieved by way of compression or injection molding, where two mating mold parts define the variable thickness throughout the entire leaflet surface being molded. Details of the manufacture of such variable thickness polymeric leaflets is detailed below.

Lastly, it is noted that all tissue-based valves, which are used in current valve replacements have a roughly uniform thickness because these tissues are harvested from animals and cannot incorporate variable thickness therein.

### 4. Anti-Leak Flap

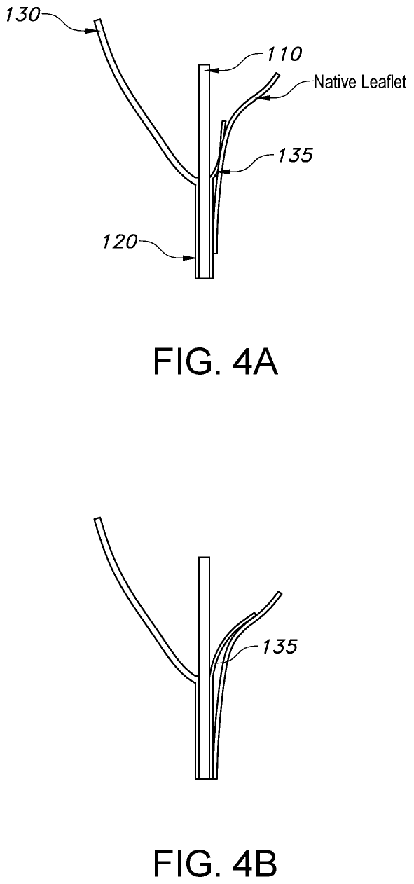

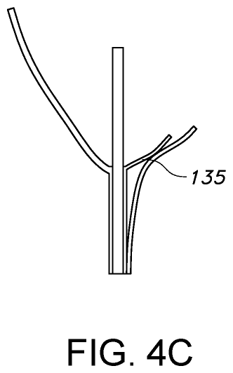

The polymeric heart valve systems described may optionally contain one or more anti-leak flaps (135), as shown in FIGS. 1A, 1C-1E, and 4A-4C. The one or more anti-leak flaps encircle the calcific leaflet level of the system. When the polymeric heart valve system is implanted in a valve (i.e., aortic valve), the anti-leak flap can be located adjacent to the sinus, native calcific leaflets, and left ventricular outflow track of the heart valve.

In some instances, the one or more anti-leak flaps include a plurality of optional slits which can improve flexibility and the ability of the anti-leak flap to cover or abut against the native calcific leaflets of the valve.

The one or more anti-leak flaps can be formed by molding and can be made of the same or a different polymer from which the polymeric sleeve and/or leaflets is formed. In most instances, the one or more anti-leak flaps, polymeric leaflets, and polymeric sleeve are formed of the same polymer/polymeric matrix. Exemplary polymers which can be used to form the one or more anti-leak flaps include, but are not limited to, thermoplastic polymers/elastomers, thermoset polymers, thermally crosslinkable polymers, elastomeric polymer biomaterials, polymers containing polysulfone(s), polyurethanes, silicones, PTFE, crosslinked poly (styrene-isobutylenese-styrene) (xSIBS), and poly (styrene-isobutylene-styrene) (SIBS). Thermoplastic elastomers (TPEs) can be formed of or contain polymyrcene, polymenthide, and poly (ϵ-decalactone). Elastomeric biomaterials can include, for example, silicones, thermoplastic elastomers, polyolefins, polydiene elastomers, poly (vinyl chloride), natural rubbers, heparinized polymers, hydrogels, polypeptides elastomers, polysiloxane-urea elastomers, and polyurethanes. As appropriate, the skilled person would be able to select conditions (i.e., temperatures, solvent(s), etc.) needed to form polymer melts or form polymer solutions of the aforementioned to be able to form the one or more anti-leak flaps during molding, as detailed further below. Typically, the one or more anti-leak flaps are molded together with the polymeric leaflets and polymeric sleeve onto the supportive frame in a single step.

The one or more anti-leak flaps can have any suitable dimension, shape, or size needed. The length of the flap extending from the surface of the polymeric sleeve can range from about 1 to 5 mm. In some instances, the thickness of any portion of the one or more anti-leak flaps can range from between about 0.01 to 100 μm, 30 to 350 μm, or about 30 to 300 μm, where the thickness can be dictated by the molding technique and the lowest limit of capable thickness which can be achieved.

The anti-leak flaps are believed to have the ability to block PVL channels by either covering the entrance to the channel, as it covers or seals gaps between the valve system and the native calcific leaflets or by filling the gaps between the valve system and the native calcific leaflets. The anti-leak flaps can seal the gaps in response to backward pressure during diastole. During systole the forward pressure is negligible, as the valve system is open, therefore the anti-leak flaps will remain stationary. As shown in FIGS. 4A-4C, the anti-leak flap is placed against the native calcific leaflets of the heart valve. The anti-leak flap may be placed in partial supra-annular position against the native calcific leaflets or in supra-annular position against native calcific leaflets of the heart. The anti-leak flaps can be useful in minimizing or preventing the risk of intra and/or paravalvular leaks.

### III. Methods of Making Polymeric Heart Valve Systems

Methods of making polymeric heart valve systems described above are detailed below.

In one non-limiting instance, a method of making can include the steps of:

(i) placing a supportive frame inside a cavity of a mold in a position suitable to facilitate flow of the polymer around the supportive frame,

wherein the mold comprises a top core, a bottom core, and one or more encasement components which encase the top and bottom cores,

wherein the top core and bottom core define a shape to form polymeric leaflets in a suitable orientation where polymeric leaflets are formed preferably in a semi-open position in a zero residual stress conformation,

optionally, wherein the shape forming the polymeric leaflets has a curvilinear profile along a circumferential axis of the polymeric leaflets being formed; and

(ii) introducing at least one polymer into the mold;

(iii) molding or casting the polymeric leaflets and a polymeric sleeve around the supportive frame to form a polymeric heart valve system,

wherein the polymeric leaflets and the polymeric sleeve are a continuum and each of the polymeric leaflets are connected to the polymeric sleeve at an attachment end; and

(iv) removing the polymeric heart valve system formed from the mold.

As previously noted, the polymeric leaflets and the polymeric sleeve form a continuum (i.e., a single continuous piece). This differs from currently known valve systems that must suture natural valve components (bovine/porcine/ovine) to the valve. The instant method allows for formation of polymeric heart valves where the polymeric leaflets formed avoid punctures because no suturing is needed because the polymeric leaflets and the polymeric sleeve are fully attached to each other.

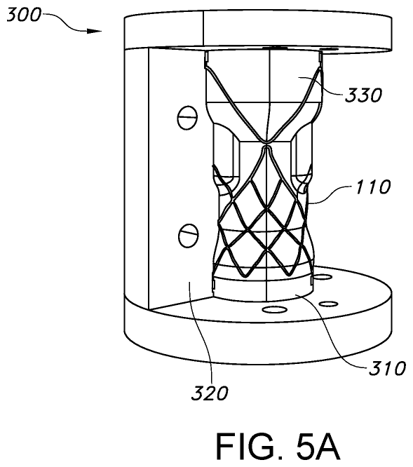

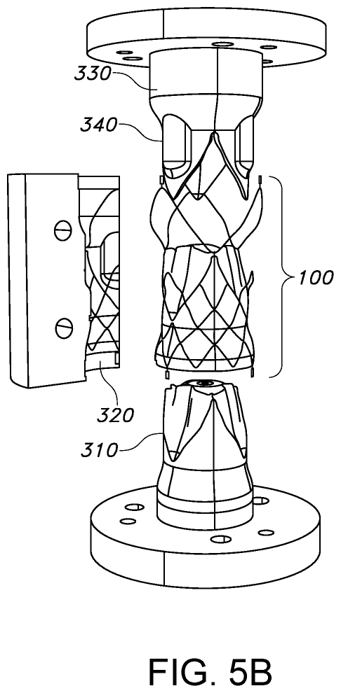

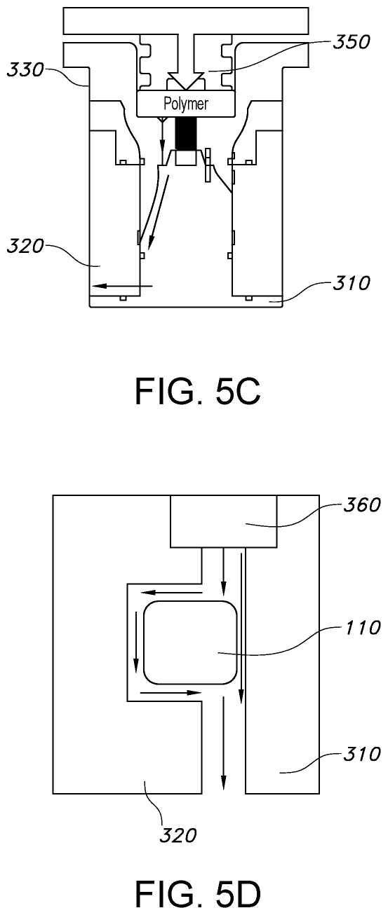

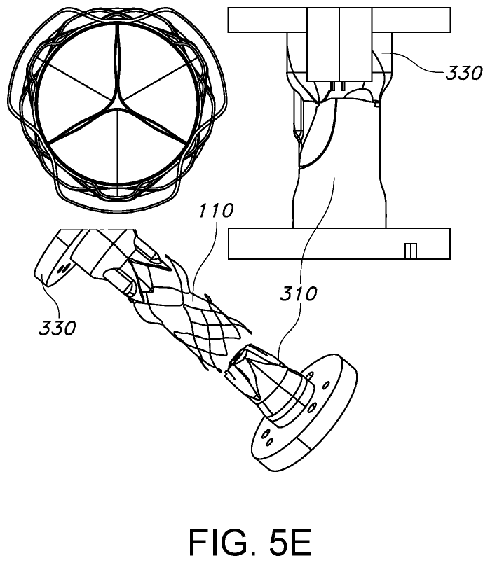

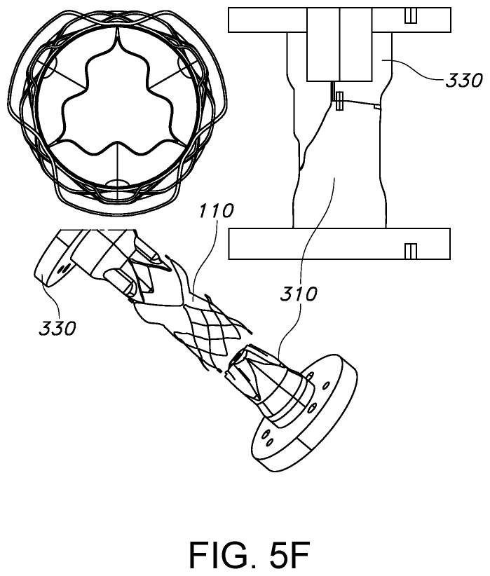

Although the method preferably produces polymeric leaflets in a semi-open position (which can reduce stresses, such as flexural stresses), the methods may also be used to produce polymeric leaflets in open or closed positions or any intermediate position. For example, FIG. 5E shows how a mold can be used to form polymeric leaflets in the closed position while FIG. 5F shows how a mold can be used to form polymeric leaflets in the semi-open position. The design of the top and bottom cores can be selected to produce polymeric leaflets of any desired position when these components are created/fabricated.

The mold used in the methods is a modular mold, where the interior of the mold and the sections that are used to form the polymeric heart valve are modular (e.g. symmetrically split to three parts in a circumferential axis) to facilitate easy extraction of the fabricated polymeric heart valve from the molding cavity. In some instances, the mold may include one or more vent holes which allow suction of air out of the mold cavity and/or the removal of excess polymer which may be introduced in step (ii). As shown in FIG. 5A, a non-limiting mold (300) having a base/ventricular core (310), encasement components (320), a top/aortic core (330), optionally having multiple axial leaflet lock registrations (340) can be placed around a supportive frame (110). The axial leaflet lock registrations (340) can be used to hold the supportive frame such as at the axial leaflet locking struts (150), shown in FIG. 1A. During step (ii) a polymer is introduced into the mold and the polymeric leaflets and the polymeric sleeve can be simultaneously molded or cast onto the supportive frame. As shown in FIG. 5B, once the polymeric heart valve system (100) is formed it can be removed during step (iv) from the mold by disassembling the mold parts. As further shown in FIG. 5B, certain parts of the supportive frame are not covered by the polymeric sleeve including the crown supporting struts and axial leaflet locking struts, as well as any anchors which may be present. Preferably, the mold is designed to not embed the crown supporting struts, axial leaflet locking struts, and any anchors present in the polymeric sleeve. In some instances, if the crown supporting struts, axial leaflet locking struts, and any anchors are covered by polymeric sleeve or any excess polymer, this may be removed thereafter.

In some instances, the mold and components thereof are formed of stainless steel, tool steel, aluminum, or any other suitable metal. The mold and components thereof may be fabricated using art known techniques, such as precision machining/CNC or 3-D metal printing techniques. The design of the mold cavities needed to produce the leaflets and polymeric sleeve on the supportive frame, having any requisite complexity of design, can be achieved in the mold parts using such precision machining/CNC or 3-D metal printing techniques or other precision machining or fabricating means including additive manufacturing. Further, the mold may include sealing elements, such as o-rings, or custom-shape and made sealing elements. For instance, if it is necessary to prevent the polymer from coating a part of the supportive frame, a sealing element with a custom shape can be made for that part of the supportive frame (e.g. from silicone or other suitable materials).

In some instances of the methods, positioning of the supportive frame into the mold cavity (in step (1)) involves the presence of an annular gap distance from the supportive frame the outer polymeric sleeve, to be formed thereon, in a size ranging from about 10 to 20 μm. To promote accurate placing of the supportive frame within the desired tolerance, arched segments (e.g. 0.1-1.0 mm thick) may be placed or present in parts of the mold components, such as the encasement components, that protrude from the outer wall of the mold and hold the supportive frame in the desired radial position. In some instances, this allows for full alignment with the polymeric heart valve centerline. Such arched segments can be spaced, for example, every 3 to 10 mm (in the long axis). In order to avoid any potential impairment of polymer flow inside the mold, slits can be cut out of the arched segments to for flow of the polymer in between the segments. In some instances, such arched segments may be retractable such that they can retracted from the mold body once the polymer has been cured sufficiently. In still other instances, instead of use of arched segments, these could be replaced with multiple pins that are spaced apart in the circumferential and longitudinal-axial directions of components of the mold. The arched segments should preferably be made of the same material as the mold so that heating and thermal expansion of the mold remains uniform throughout. Nevertheless, use of combinations of various metals or polymers can be used to form the arched segments or mold components that do not experience mechanical loading. As an example, a plunger to apply force/pressure to the polymer can be made of one type of steel (i.e., tool steel) while components with surfaces that are in contact with the valve/stent being molded/cast can be made of a different kind of steel (i.e., stainless steel). Moving parts, such as a plunger, may benefit from being made of harder steel to minimize potential wear and particle leaching.

In some instances of the methods, the mold, prior to step (i) or (ii) may be cleaned and prepared (chemically and/or mechanically). In some instances, the mold is heated during steps (ii) or (iii) to a suitable temperature, such as to ensure ease of flow of the polymer through the mold. Such heating temperatures can range from about 100° C. to 350° C., and subranges within. The molding or casting of step (iv) can involve curing the polymer, where after curing is complete (a function of temperature and time) the mold is allowed to cool down, then the mold is opened and the cured polymeric heart valve removed. In some instances of the methods, a vacuum can be applied into the mold to remove air bubbles from the polymer introduced into the mold cavity which cured to produce the molded or cast polymer. The ability to produce a vacuum may be integrated into the mold itself. In some instances, when polymer curing is completed, the vacuum (if applied) is released, and the mold is partially opened up while still heated/hot and the internal mold parts that “wrap” the cured polymeric heart valve are then allowed to cool down, either slowly to room temperature, or quickly by immersion into water.

In some instances, as the polymer formed by, for example, compression/transfer/injection molding is cured under heat and pressure, the resulting polymer becomes denser and less porous. Manipulation of the outer surface roughness can assist in adhesion to the native tissue. Another potential advantage of such rough surface can be lower friction during crimping/collapsing/compacting of the valve system for deployment into a subject.

In some instances, the formed polymeric valve system can be carefully extracted from the mold in step (iv): by separation from the mold using, for example, a spatula, air pressure, liquid pressure (e.g. isopropyl alcohol), immersion in a solvent (e.g. alcohols, such as ethanol or isopropyl alcohol) optionally with sonication. Following step (iv), the methods described may also involve a further step of removing of any excess polymer from the valve which is not desired, such as by cutting, laser cutting, ultrasonic cutting, thermal cutting, or other cutting means. The methods may additionally involve a further step of cleaning the formed polymeric valve after removal from the mold in suitable solvent(s) optionally with ultrasonication and then drying the polymeric valve thereafter. Following step (iv), the method may further include a step of conformally coating at least part or all of the outer surfaces of the polymeric sleeve with a gel paving material, such as made from a hydrogel. In some instances, the gel paving material may be applied via forming gels as films and wrapping the external surface of the polymeric sleeve and/or polymeric leaflets with the gel paving material, allowing it to dry, thereby constriction boding to the surface. In some other instances, the external surface of the polymeric leaflets and/or polymeric sleeve may be doped with a layer of suitable adhesive to which the gel paving material-either wet or in desiccated form is applied. In still other cases, the external surface of the polymeric leaflets and/or polymeric sleeve may be coated with a photoreactive catalyst—e.g. eosin or riboflavin, and the gel formed as a layer via photocatalysis as described by Slepian in U.S. Pat. No. 6,290,729. The thickness of the gel paving material, in desiccated form, may range from between about 0.5 to 5 mm.

In some instances of the methods, a plunger or other means of applying pressure may be used when the polymer is introduced in step (ii). The polymer may be introduced through, for example, an opening in the top/aortic core of the mold and a plunger may be used to apply pressure to the polymer to ensure it spreads throughout the cavities of the mold. Suitable pressures which can be used to flow the polymer during steps (ii) and (iii) can range from between about 1 to 10 tons, and sub-ranges or individual values contained within. For example, FIG. 5C shows a cross-sectional representation of a mold where a polymer is applied under pressure by way of a plunger (350). As shown in FIG. 5D the polymer flows between cavities in the encasement components and core components in order to embed the supportive frame (110) in the polymeric matrix.

In some instances, the polymeric sleeve formed further includes one or more anti-leak flaps which are formed by using a mold that defines the anti-leak flaps and forms the anti-leak flaps, when the polymer is molded/cast in step (iii).

In some cases, one or more releasing agents may be applied to the mold surfaces prior to step (ii) in order to facilitate removal of the polymeric heart valve system in step (iv). Suitable mold releasing agents include solvent-based, water-based agents, silicone agents, or resin-based agents, which may be sacrificial or semi-permanent. These agents can be sprayed onto the surfaces at room temperature or with heating (of about 50 to 200° C. or about 100 to 150° C.) in a single or multiple layers. Various types of mold release agents are commercially available and known to those skilled in the art.

In some instances, the molding or casting of the at least one polymer in step (iii) is performed by a compression molding process, an injection molding process, a dip coating process, or a transfer molding process. Compression molding, for example, relies on part of the shape setting mold to move and compress the polymer, cause pressure and flow into the other mold cavities. Injection molding, for example, relies on an extruder system to heat and pressurize the polymer, causing it to flow into the mold cavities. Transfer molding, for example, is similar to injection molding but instead of an extruder generating the pressure, a plunger system is pressurized (compressed) into a cavity of heated polymer and injected into the cavities of the mold. Such processes and conditions for such processes are known to those of ordinary skill in the art. In one non-limiting example of the method, during step (ii) a polymer (e.g. pellets or powder form) is placed into the mold, if compression molding, or into a container adjacent to the mold, if transfer or injection molding is employed, and the mold is heated to a temperature that would make the raw polymer pliable in a fluid gel-like form.

Exemplary polymers, or blends thereof, which can be used to form the polymeric leaflets and polymeric sleeve in the methods described include, but are not limited to, thermoplastic polymers/elastomers, thermoset polymers, thermally crosslinkable polymers, elastomeric polymer biomaterials, polymers containing polysulfone(s), polyurethanes, silicones, PTFE, crosslinked poly (styrene-isobutylenese-styrene) (xSIBS), and poly (styrene-isobutylene-styrene) (SIBS). Thermoplastic elastomers (TPEs) can be formed of or contain polymyrcene, polymenthide, and poly (ϵ-decalactone). Elastomeric biomaterials can include, for example, silicones, thermoplastic elastomers, polyolefins, polydiene elastomers, poly (vinyl chloride), natural rubbers, heparinized polymers, hydrogels, polypeptides elastomers, polysiloxane-urea elastomers, and polyurethanes. As appropriate, the skilled person would be able to select conditions (i.e., temperatures, solvent(s), etc.) needed to form polymer melts or form polymer solutions of the aforementioned for use in step (ii) and during molding/casting step (iii). In some instances, thermoplastic polymers can be chosen when instances of dip coating are used to form the systems described and thermoset polymers can be chosen when instances of compression, transfer, or injection molding are used to form the systems described. In still other instances, a combination of both dip coating of the polymeric sleeve and to attach them to previously molded polymeric leaflets can be used.

Details of the supportive frame, polymeric sleeve, polymeric leaflets, and anti-leak flap which are formed according to the methods described to provide polymeric heart valve systems are given below.

### 1. Supportive Frame

In some instances, the supportive frame used in the methods is formed of an armature or scaffold-like construct formed from a plurality of connected struts, wherein each strut is connected to one or more other struts via a joint. In some other instances, the armature or scaffold-like construct may be in the form of a cage having tubular or wire-like elements configured in a stent-like configuration. The armature or scaffold-like construct is expandable from its crimped, compacted, or collapsed back up to its size, as formed, or any desired size in between. Preferably, the armature or scaffold-like construct is an expandable stent which is self-expandable or otherwise requires active expansion by balloon expansion or heat expansion. Expandable stents are plastically deformed with mechanical energy, such as by balloon expansion, into an “opened” or deployed shape. Self-expandable stents can be thermally fixed or shape set after expansion. Such expandable stents and materials for making such stents are known in the art. Suitable materials, dimensions, characteristics, and methods of manufacturing the supportive frame are described above. Further, as previously noted, the supportive frame may include optional components including one or more anchors and/or one or more indentations which are preferably not covered/coated by any polymer or polymeric matrix during or following steps (ii), (iii), and (iv), or where any polymer thereon can be removed after these steps are performed.

### 2. Polymeric Sleeve

In some instances, the thickness of the embedding polymeric matrix forming the polymeric sleeve can have any suitable thickness thereon. The thickness of the extraluminal polymeric matrix material covering the supportive frame can, for example, when formed by compression molding or other molding techniques have a thickness in a range from about 0 to 500 microns in the circumferential direction and/or a thickness ranging from about 0 to 400microns in the radial direction of the struts or wire-like elements of the supportive frame. In some instances, an average thickness of about 400 microns in the circumferential direction (i.e, about 200 microns are on each side of the strut) is present. Suitable materials, dimensions, and characteristics of the polymeric sleeve which can be formed according to the methods herein are described above. As previously noted, in some instances, the outer surface may be coated with a gel paving material subsequent to formation of the polymeric sleeve and removal from the mold.

### 3. Polymeric Leaflets

As noted above, the polymeric leaflets of the system can be achieved, for example, by forming the polymeric leaflets and polymeric sleeve (onto the supportive frame) at the same time during the molding process (i.e., single manufacturing step) whereby they form a single polymeric component containing both the polymeric leaflets and polymeric sleeve. The polymeric leaflets can be formed of polymers as previously specified, and are typically made of the same polymer as the polymeric sleeve.

However, in less preferred instances of the methods, polymeric leaflets may be formed/molded as a first component and then the leaflets and a supportive frame can be dip coated into a suitable polymer melt or solution to form the polymeric sleeve. During such instances, the polymer melt or solution forms the polymeric sleeve over the supportive frame and simultaneously attaches to the polymeric leaflets in the mold. Thus, the leaflets become an inseparable part of the formed polymeric sleeve.

The polymeric leaflets forming the valve can have any suitable dimension, shape, or size needed. In most instances, the valve system includes three polymeric leaflets which each have a curvilinear or wavy profile in a circumferential axis and are preferably formed/molded in a semi-open conformation. The design of the top and bottom cores can be selected to produce polymeric leaflets of any desired position when these mold components are created/fabricated; see FIG. 5E and 5F.

The polymeric leaflets can each independently have uniform or non-uniform thicknesses. However, in some preferred instances, the polymeric leaflets each independently have a non-uniform (i.e., variable) thickness across any cross-sectional direction of the leaflet. Such variable thickness can be imparted by the selection of the design of the top and bottom cores of the mold in order to produce polymeric leaflets of any desired thickness and having any desired variability in thickness therein, when these mold components are created/fabricated.

### 4. Anti-Leak Flap

The polymeric heart valve systems formed according to the methods above can include one or more anti-leak flaps that encircle the calcific leaflet level of the system. The anti-leak flaps are defined by the mold, which can be designed to produce such flaps on the polymeric sleeve. The anti-leak flaps can be formed of polymers as previously specified, and are typically made of the same polymer as the polymeric sleeve and/or polymeric leaflets. In some instances, the one or more anti-leak flaps include a plurality of optional slits which can improve flexibility and the ability of the anti-leak flap to cover or abut against the native calcific leaflets of the valve.

### IV. Uses of Polymeric Heart Valve Systems

The polymeric heart valve systems described above can be used to replace organ valves in a subject, such as human. In some instances, the polymeric heart valve systems can replace defective aortic heart valves. Alternatively, the polymeric heart valve system may be utilized to replace other heart valves; or serve as an extracardiac valve, e.g. in the aorta or other arteries. In still other instances, the polymeric heart valve system is not limited to use only in the heart or structures therein and may be used in veins; or other luminal structures organs, or organ components of the body of a subject.

The disclosed artificial polymeric valve systems are expected to have a lower occurrence of wear and tear durability failures compared to tissue-based TAVR devices. The disclosed artificial polymeric valve systems are expected to have a lower occurrence of calcific growth within the TAVR polymeric leaflets compared to tissue-based TAVR devices. In some instances, the heart valve systems described herein have a reduction in calcification within the TAVR polymeric leaflets of at least about 10%, 20%, 30%, 40%, 50%, 60%, 70%, 80%, 90%, 95%, 99%, or greater, compared to the native valve. The heart valve system's calcification susceptibility can be determined using a suitable known protocol, such as for example, using an in vitro protocol (Boloori, Z. P., et al., Mater Sci Eng C Mater Biol Appl 35:335-340, 2014) utilizing an accelerated wear testing (AWT) 50 setup with a pro-calcific/phosphorus compound (Golomb G, et al., Biomaterials 12:397-405, 1991) for at least fifty million valve operation cycles. See also Rotman, O. M., et al., ASAIO J, 66 (2), pp. 190-198 for a detailed discussion on durability and stability testing of polymeric transcatheter valves.

In some instances, the polymeric heart valve systems can be implanted in a subject in need thereof to replace a defective valve. A non-limiting exemplary method can include the steps of:

(1) inserting a polymeric heart valve system in a crimped, collapsed, or compacted state into the subject in need thereof through an artery;

(2) delivering the polymeric heart valve system to a defective valve in the subject in need thereof;

(3) implanting the polymeric heart valve system by expanding the crimped, collapsed, or compacted polymeric heart valve system into an expanded state which localizes or fixes the polymeric heart valve system at the defective valve and replaces the function of the defective valve.

In some instances of the above method, the subject is a human and the defective valve is a defective aortic valve. In some instances, the insertion in step (1) involves the use of a delivery catheter or similar extendable member or delivery system. In some cases, the polymeric heart valve system is crimped, collapsed, or compacted immediately prior to use and then inserted into a delivery catheter.

As noted above, the polymeric heart valve system is not limited to use only in the heart or structures therein. Accordingly, in some instances, the system may be used in a method of treating a subject in need thereof, the method including the steps of:

(1′) inserting a polymeric valve system, as described herein, in a crimped, collapsed, or compacted state into the subject in need thereof;

(2′) delivering the polymeric valve system to an artery, vein, or luminal structure organ in the subject in need thereof;

(3′) implanting the polymeric valve system by expanding the crimped, collapsed, or compacted polymeric heart valve system into an expanded state, which localizes or fixes the polymeric heart valve system at the artery, vein, or luminal structure organ.

The disclosed polymeric heart valve systems and methods can be further understood through the following numbered paragraphs.
1. A polymeric heart valve system comprising:

a supportive frame and a plurality of polymeric leaflets;

wherein the supportive frame comprises a plurality of openings, and wherein the supportive frame is embedded in a polymeric matrix in the form of a polymeric sleeve;
wherein each of the plurality of polymeric leaflets are a continuum of the polymeric sleeve without sutures, and each of the plurality of polymeric leaflets are connected to the polymeric sleeve at an attachment end; and

wherein the plurality of polymeric leaflets are able to open and close at an operative end, wherein when the plurality of leaflets are in the closed position, the operative ends of the leaflets abut each other.
2. The polymeric heart valve system of paragraph 1, wherein the supportive frame is formed from a plurality of connected struts each connected to one or more other struts via a joint.
3. The polymeric heart valve system of paragraph 2, wherein the plurality of connected struts each comprise anchors and/or wherein the plurality of connected struts and/or the one or more joints each comprise indentations and/or openings.
4. The polymeric heart valve system of paragraph 2, wherein the plurality of connected struts each comprise one or more surfaces having surface features ranging from smooth and featureless to others having micro and/or macro asperities and the one or more surfaces are not exposed in order to prevent risk of thrombus formation.
5. The polymeric heart valve system of any one of paragraphs 1 to 4, wherein the supportive frame further comprises a crown region in which the polymeric matrix is not located, optionally wherein the crown region comprises one or more anchors.
6. The polymeric heart valve system of any one of paragraphs 1 to 5, wherein the supportive frame is in the form of an armature or scaffold-like construct which is expandable.
7. The polymeric heart valve system of any one of paragraphs 1 to 6, wherein the supportive frame is made of a metal or metal alloy selected from the group consisting of spring steel, stainless steel, platinum, tantalum alloys, cobalt chromium, NiTi, NiTiCo, NiTiCr, NiTiCu, and NiTiNb.
8. The polymeric heart valve system of any one of paragraphs 1 to 7, wherein the polymeric sleeve is made of a biocompatible, hemocompatible, and mechanically stable polymer.
9. The polymeric heart valve system of any one of paragraphs 1 to 7, wherein the polymeric sleeve comprises one or more polymers selected from the group consisting of thermoplastic polymers/elastomers, thermoset polymers, thermally crosslinkable polymers, elastomeric polymer biomaterials, polymers containing polysulfone(s), PTFE, crosslinked poly (styrene-isobutylenese-styrene) (xSIBS), poly (styrene-isobutylene-styrene) (SIBS), polymyrcene, polymenthide, and poly (ϵ-decalactone), silicones, polyolefins, polydiene elastomers, poly (vinyl chloride), natural rubbers, heparinized polymers, hydrogels, polypeptides elastomers, polysiloxane-urea elastomers, and polyurethanes.
10. The polymeric heart valve system of any one of paragraphs 1 to 9, wherein the polymeric sleeve further comprises one or more anti-leak flaps thereon.
11. The polymeric heart valve system of any one of paragraphs 1 to 10, wherein the polymeric sleeve further comprises a gel paving material on one or more outer surfaces of the polymeric sleeve.
12. The polymeric heart valve system of any one of paragraphs 1 to 11, wherein the polymeric sleeve has a thickness of about 0 to 500 μm.
13. The polymeric heart valve system of any one of paragraphs 1 to 7, wherein the polymeric leaflets comprise one or more polymers selected from the group consisting of thermoplastic polymers/elastomers, thermoset polymers, thermally crosslinkable polymers, elastomeric polymer biomaterials, polymers containing polysulfone(s), PTFE, crosslinked poly (styrene-isobutylenese-styrene) (xSIBS), poly (styrene-isobutylene-styrene) (SIBS), polymyrcene, polymenthide, and poly (ϵ-decalactone), silicones, polyolefins, polydiene elastomers, poly (vinyl chloride), natural rubbers, heparinized polymers, hydrogels, polypeptides elastomers, polysiloxane-urea elastomers, and polyurethanes.
14. The polymeric heart valve system of any one of paragraphs 1 to 13, wherein each of the leaflets in the plurality of leaflets comprises variable thickness having a center line of symmetry, where in a given cross-section each of the leaflets has at least two or more thicknesses.
15. The polymeric heart valve system of paragraph 14, wherein each of the leaflets has a minimum thickness in a range from about 50 to 200 μm.
16. The polymeric heart valve system of paragraph 14, wherein each of the leaflets has a maximum thickness in a range from about 200 to 600 μm.
17. The polymeric heart valve system of any one of paragraphs 1 to 16, wherein the polymeric sleeve is formed from the same polymeric material as the polymeric leaflets, or from a different polymeric material than the polymeric leaflets.
18. The polymeric heart valve system of any one of paragraphs 1 to 17, wherein the supportive frame and/or supportive sleeve comprise radiopaque materials.
19. A method of making a polymeric heart valve system, the method comprising the steps of:

(i) placing a supportive frame inside a cavity of a mold in a position suitable to facilitate flow of the polymer around the supportive frame,

wherein the mold comprises a top core, a bottom core, and one or more encasement components which encase the top and bottom cores,

wherein the top core and bottom core define a shape to form polymeric leaflets in a suitable orientation wherein the polymeric leaflets are formed preferably in a semi-open position having a zero residual stress conformation,

optionally, wherein the shape forming the polymeric leaflets has a curvilinear profile along a circumferential axis of the polymeric leaflets being formed; and

(ii) introducing at least one polymer into the mold;

(iii) molding or casting the polymeric leaflets and a polymeric sleeve around the supportive frame to form a polymeric heart valve system,

wherein the polymeric leaflets and the polymeric sleeve are a continuum and each of the polymeric leaflets are connected to the polymeric sleeve at an attachment end; and

(iv) removing the polymeric heart valve system formed from the mold.
20. The method of paragraph 19, wherein the one or more encasement components further comprising one or more arched segments or pins and/or one or more sealing elements;

wherein the one or more arched segments or pins hold the supportive frame.
21. The method of any one of paragraphs 19 or 20, wherein the method includes a mold cleaning step prior to step (i).
22. The method of any one of paragraphs 19 to 21, wherein the mold is heated during steps (ii) and/or (iii) to a temperature ranging from about 100° C. to 350° C.
23. The method of any one of paragraphs 19 to 22, wherein step (ii) comprises applying pressure in a range from about 1 to 10 tons by way of a plunger to spread the polymer through the mold.
24. The method of any one of paragraphs 19 to 23, wherein step (iii) is performed by a compression molding process, transfer molding process, or injection molding process.
25. The method of any one of paragraphs 19 to 23, wherein step (iii) is performed by dip coating process.
26. The method of any one of paragraphs 19 to 25, further comprising, prior to step (i), applying one or more release agents to the surfaces of the mold to facilitate removal of the polymeric heart valve system after formation.
27. The method of any one of paragraphs 19 to 26, wherein the supportive frame is formed from a plurality of connected struts each connected to one or more other struts via a joint.
28. The method of any one of paragraphs 19 to 25, wherein the supportive frame is in the form of an armature or scaffold-like construct which is expandable.
29. The method of any one of paragraphs 19 to 28, wherein the supportive frame is made of a metal or metal alloy selected from the group consisting of spring steel, stainless steel, platinum, tantalum alloys, cobalt chromium, NiTi, NiTiCo, NiTiCr, NiTiCu, and NiTiNb.
30. The method of any one of paragraphs 19 to 29, wherein the polymer is selected from one or more of the group consisting of thermoplastic polymers/elastomers, thermoset polymers, thermally crosslinkable polymers, elastomeric polymer biomaterials, polymers containing polysulfone(s), PTFE, crosslinked poly (styrene-isobutylenese-styrene) (xSIBS), poly (styrene-isobutylene-styrene) (SIBS), polymyrcene, polymenthide, and poly (&-decalactone), silicones, polyolefins, polydiene elastomers, poly (vinyl chloride), natural rubbers, heparinized polymers, hydrogels, polypeptides elastomers, polysiloxane-urea elastomers, and polyurethanes.
31. The method of any one of paragraphs 19 to 30, wherein the polymeric sleeve further comprises one or more anti-leak flaps thereon.
32. The method of any one of paragraphs 19 to 31, wherein the method further comprises a step of coating the polymeric sleeve, after step (iv) with a gel paving material onto one or more outer surfaces of the polymeric sleeve.
33. The method of any one of paragraphs 19 to 32, wherein each of the polymeric leaflets comprises variable thickness across cross-sections of each of the polymeric leaflets running from an attachment end to an operative end thereof.
34. The method paragraph 33, wherein each of the polymeric leaflets has a minimum thickness in a range from about 50 to 200 μm.
35. The method of any one of paragraphs 33 or 34, wherein each of the polymeric leaflets has a maximum thickness in a range from about 200 to 600 μum.
36. The method of any one of paragraphs 19 to 35, wherein step (i) comprises placing the supportive frame inside the cavity of the mold while inducing the supportive frame to comprise a degree of oversizing during molding or casting step (iii).
37. A polymeric heart valve system comprising:

a supportive frame and a plurality of polymeric leaflets;

wherein the supportive frame comprises a plurality of openings, and wherein the supportive frame is embedded in a polymeric matrix in the form of a polymeric sleeve;

wherein each of the plurality of polymeric leaflets are a continuum of the polymeric sleeve without sutures, and each of the plurality of polymeric leaflets are connected to the polymeric sleeve at an attachment end; and

wherein the plurality of polymeric leaflets are able to open and close at an operative end, wherein when the plurality of leaflets are in the closed position, the operative ends of the leaflets abut each other;

wherein the polymeric heart valve system is formed according to the method of any one of claims 17-36.
38. A method of replacing a defective valve in a subject in need thereof, the method comprising the steps of:

(1) inserting a polymeric heart valve system of any one of paragraphs 1-18 in a crimped, collapsed, or compacted state into the subject in need thereof through an artery;

(2) delivering the polymeric heart valve system to the defective valve in the subject in need thereof;

(3) implanting the polymeric heart valve system by expanding the crimped, collapsed, or compacted polymeric heart valve system into an expanded state, which localizes or fixes the polymeric heart valve system at the defective valve and replaces the function of the defective valve.
39. The method of paragraph 38, wherein the defective valve is an aortic valve.
40. The method of any one of paragraphs 38-39, wherein the polymeric heart valve system in the crimped, collapsed, or compacted state is inserted during step (i) via a delivery catheter.
41. The method of any one of paragraphs 38-40, wherein the polymeric heart valve system is crimped, collapsed, or compacted state prior to step (i).
42. The method of any one of paragraphs 38-41, wherein expanding of the crimped, collapsed, or compacted polymeric heart valve system in step (3) is performed by balloon expansion or heat expansion.
43. A method of treating a subject in need thereof, the method comprising the steps of:

(1′) inserting a polymeric valve system of any one of paragraphs 1-18 in a crimped, collapsed, or compacted state into the subject in need thereof;

(2′) delivering the polymeric valve system to an artery, vein, or luminal structure organ in the subject in need thereof;

(3′) implanting the polymeric valve system by expanding the crimped, collapsed, or compacted polymeric heart valve system into an expanded state, which localizes or fixes the polymeric heart valve system at the artery, vein, or luminal structure organ.

### EXAMPLES

### Methods

A polymeric heart valve system features a unique design with suture-less over molding to create the polymeric leaflets and skirt/sleeve in a seamless single manufacturing step. The stent (supportive frame) is registered within the mold and the polymer is cast to create the leaflet design and sleeve with wrapped features around the struts of the stent. It additionally reduces the risk of calcification and thrombus formation focal points that may occur where sutures are sewn and affixed to the tissue. The continuous wrap of the polymer around the stent and the adhesion of the polymer to the metallic frame allows even distribution of leaflet forces during valve operation.

The polymeric heart valve system for TAVR can be formed by molding a cross-linkable poly (styrene-block-IsoButylene-block-Styrene) (SIBS) material onto a supportive frame and forming a polymeric sleeve thereon. The xSIBS material can be used formed with an intra-molding cross-linking technology (Rotman, O. M., et al., ASAIO J, 66 (2), pp. 190-198; Rotman, O. M., et al., Ann Biomed Eng, 47 (1), pp. 113-125; Kovarovic, B. J., et al., Artif Organs, 45 (4), pp. E41-E52; and Ghosh, R. P., et al., 2020, “Numerical evaluation of transcatheter aortic valve performance during heart beating and its post-deployment fluid-structure interaction analysis,” Biomechanics and Modeling in Mechanobiology. 10.1007/s10237-020-01304-9). By cross linking the SIBS polymer (xSIBS) customized for a desired valve design and use, it is possible to achieve an intrinsic leaflet strength (Claiborne, T. E., et al., Asaio j, 57 (1), pp. 26-31;Claiborne, T. E., et al., J Biomech Eng, 135 (2), p. 021021.10.1115/1.4023235; and Piatti, F., et al., J Biomech, 48 (13), pp. 3641-3649) and to design the polymeric leaflet shape for reduced thrombosis using the device thrombogenicity emulation (DTE) technique (Claiborne, T. E., et al., Asaio j, 59 (3), pp. 275-283). Further, the leaflets can be scaled to larger sizes and further designed for reducing the stresses during valve opening and closing, as well as the crimping stresses experienced by TAVR devices before delivery in a crimped state.

### Stent Frame for TAVR

A major challenge in the design and manufacture of polymeric TAVR devices is achievement of the proper deployment size, radial force, and avoidance of plastic deformation during crimping, all while minimizing the volume of the crimped stent and polymer material. Unlike tissue valves, polymeric materials are incompressible, and the total volume must be able to fit in the crimped annular volume (i.e., 18F). Additionally, with the sutureless design, the entire polymer and metal strut needed to be considered so that during crimping, the metal stent joints and their bending patterns do not place extreme stresses on the polymer.

A series of implicit finite element analysis (FEA) simulations, Abaqus 2020 (Dassault Systemes, Vélizy-villacoublay, France), were conducted on single strut designs. The strut would be representative of the base cell of the final stent frame (i.e., supportive frame) with four mirrored struts completing the cell. The strut designs were meshed with C3D8R elements with enhanced hourglass control and a mesh convergence/sensitivity analysis was conducted. A circumferential surface was radially displaced contacting the outer surface and crimping the single strut to below the desired 18 F specification. Radial symmetry and radial contact planes maintained the correct motion and stresses within the strut. The nitinol material of the strut was assumed to be superelastic (Nematzadeh, F., et al., 2012, Scientia Iranica, 19 (6), pp. 1564-1571) and generated from the stent manufacturers material testing. The strut designs were parametrically varied, and the design was varied to produce the greatest radial force within the deployment range of the device (between 20-25 mm diameter for a 27 mm polymeric heart valve system), the lowest crimping strain or at least below the yield limit of 10% strain for nitinol, while maintaining the lowest surface area or volume.

A final stent (supportive frame) shape utilizing the modeled strut and an initial laser cutting profile was created to include the shape and attachment of the polymeric leaflets. The laser cut profile was generally cut in a smaller stock nitinol tubing, expanded on a mandrel in multiple steps and shape set to create the final stent (supportive frame) product. This process was simulated in FEA to create intermediate mandrel shapes and achieve a more consistent expanded shape required for the sutureless molding process. The final stent (supportive frame) design was further tested in patient-specific and idealized simulations to confirm that deployment, anchoring and cyclic fatigue strains were within the desired limits.

### Polymeric Leaflet Shape and Thickness for TAVR

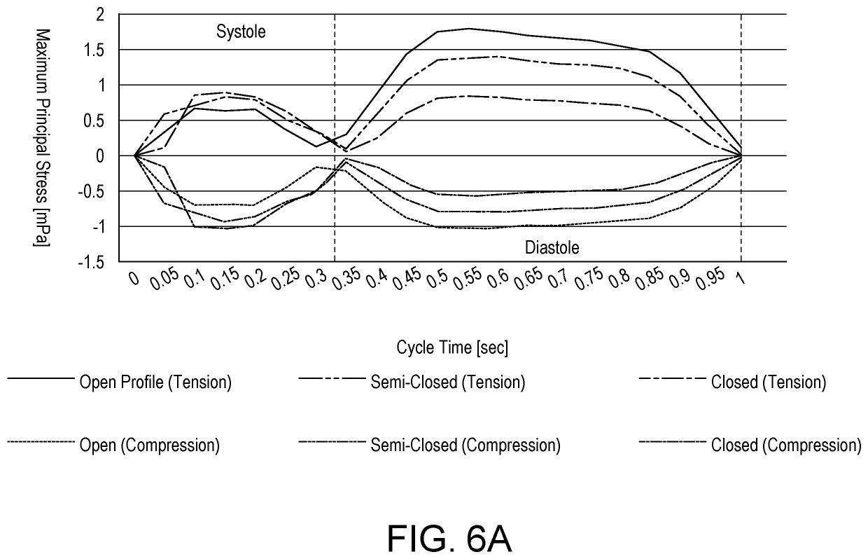

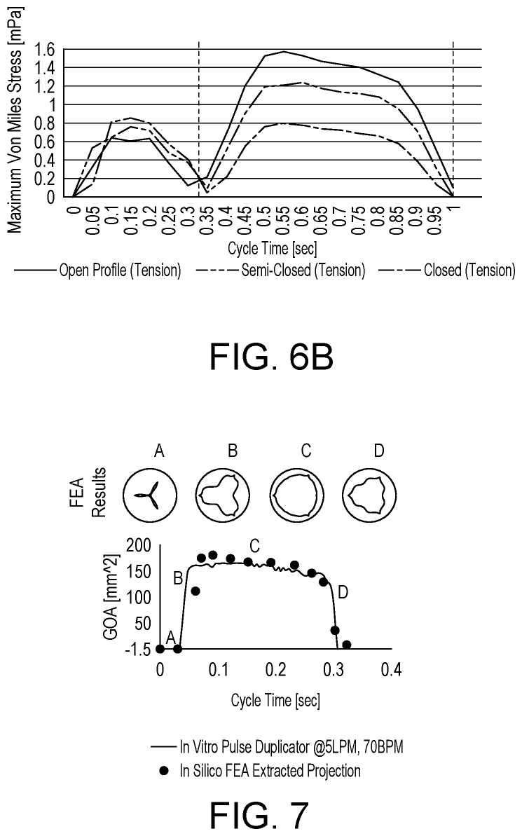

With the creation of the final stent (supportive frame) design, the shape of the polymeric leaflet attachment region was defined, and the final polymeric leaflet shape was determined. The polymeric leaflet shape design process was a multistep process utilizing explicit FEA simulations. The process changes the initial free stress profile of the polymeric leaflets (cast/molded shape), as well as varying the thickness within the polymeric leaflet. This iterative design approach reduces the peak and average stresses within the polymer leaflet material over a typical cardiac cycle with a typical physiological normotensive pressure gradient waveform (Rotman, O. M., et al., Ann Biomed Eng, 47 (1), pp. 113-125; Kovarovic, B. J., et al., Artif Organs, 45 (4), pp. E41-E52; and Standardization, I. O. f., 2021, “ISO 5840-3:2021 Cardiovascular implants-Cardiac valve prostheses-Part 3: Heart valve substitutes implanted by transcatheter techniques,” p. 57). Systolic opening pressure was varied in the testing scenarios, and resulted in using an 8 mmHg average systolic gradient to open the valve, which is typical for a high-performance valve (Johnson, N. P., et al., European Heart Journal, 39 (28), pp. 2646-2655). In this example, a normotensive diastolic gradient was found, with 100 mmHg peak back pressure, but other pathological conditions were additionally tested in silico. The pressure gradient was applied to the aortic side of the polymeric leaflets and the simulation was conducted with all three polymeric leaflets contacting during diastole. Mesh and temporal convergence/sensitivity analysis was conducted on the leaflets with the same pressure gradient waveform, and the leaflets maintained the same C3D8R elements with enhanced hourglass control with three layers across the thickness of the leaflet. The polymeric material was modelled as an isotropic hyperelastic material (Arruda-Boyce Model) based on uniaxial tensile data. Results are shown in FIGS. 6A and 6B.

The first step was to design a polymeric leaflet with an initial configuration similar to typical TAVR and surgical valve devices, with polymeric leaflets in an almost closed/diastolic configuration. Another consideration for polymeric leaflets was that the molding configuration must be considered in the design. The initial opening and closing cycle was simulated with uniform 300 μm leaflet thickness. The polymeric leaflet shape was extracted, and the simulation was restarted after zeroing the stresses, effectively simulating a design with the free stress state of the leaflets. The cyclic stresses were analyzed, and a final free stress profile was selected before varying the leaflet thickness.

In order to vary the thickness of the polymeric leaflet, the stresses and mesh were passed into a custom MATLAB algorithm (MathWorks, Natick, MA) scripted for reducing the stresses between a range of desired upper and lower stress limits. The thickness was varied along the entire volume of the leaflet, accentuating regions of higher stresses while reducing the overall volume of the leaflets (vital for the polymer crimping). The output design was re-tested in silico while undergoing the same cardiac cycle conditions, and the resultant stresses compared. These two steps of minimizing the free stress state and the thickness were iterated until an acceptable polymeric leaflet shape was determined.

### Results

A final stent (supportive) frame design resulting from the simulations includes a strut shape which is curvilinear, and includes variable circumferential thicknesses along the length of the strut. FIG. 1B is representative of the final design. Overall, the thickness of the strut was about 150-250 μm circumferential and 300 μm radial. This larger sweeping curved shape allowed bending motions during crimping to occur over the length of the strut; thus bending was not constrained to the joint region. The larger bending curvature allows for more polymer material to be cast in the joint region without risking increased shearing stresses against the polymer material that may lead to damage during crimping. Importantly, the strut is able to produce higher radial forces as a function of the volume or area of the strut. The open joint angle of the strut allowed for higher radial force in the desired deployment region between 20-25 mm (typical desired range of oversizing for this exemplary TAVR device).

The polymeric heart valve system, as designed, also achieves variable radial force over the length of the device with higher force, aiding in anchoring against the aortic annulus and calcific leaflets, which can lower the radial forces in the left ventricular outflow track (LVOT), thus reducing CCA risk. The crown region can contact the sinotubular junction of the aorta and preferably also has lower radial forces to avoid damage to the aorta, achieved with three longer contacting joints incorporated into the design. Lower radial force in the LVOT section is achieved with longer struts and the additional radial flaring aids in sealing against PVL. Lastly, one or more uncoated locking struts can be placed in the calcific leaflet level region of the TAVR leaflet to aid in axially locking the TAVR device and resisting device migration. This locking strut feature can remain uncoated so that minimal polymer remains behind the leaflets, thus reducing the stagnant flows that may increase HALT rates.

The final stent (supportive frame) design was manufactured, and in vitro measurements of the crimping radial force were conducted and compared to the in silico simulation results, confirming the expected results. Additional in silico simulations for fatigue analysis of the stent were conducted by placing a cyclic radial displacement and force due at the polymeric leaflet commissures, while ensuring that strain amplitudes remained below 0.5%. Complex crimping simulations with the sutureless polymer sleeve and polymeric leaflets were additionally conducted prior to finalizing the stent design.

### 27 mm Polymeric Leaflets:

The target goal for the polymeric leaflets was to maintain a peak stress of <2 MPa. A comparison of three opening profiles (not shown) indicated that there was a tradeoff with a 27 mm leaflet size, with more open profiles tending to have higher diastolic stresses and lower systolic stresses, and vice versa for more closed configuration profiles. A more closed profile was selected for the polymeric leaflets since the initial uniform profile was already able to achieve <1.8 MPa stresses. The presented von Mises stresses are applicable to this analysis since the xSIBS material was assumed isotropic and should be compared to the 5 MPa yield stress of the material (Claiborne, T. E., et al., ASAIO journal (American Society for Artificial Internal Organs: 1992), 59 (3), pp. 275-283). Additionally, the larger leaflet sizes benefit from the decreased closing flow achieved by the more closed configuration profile, as the leaflets close faster.

Following the selection of the opening profile, the design was modified to include a variable polymeric leaflet thickness (not shown). A benefit of variable thickness leaflets includes achieving a significant reduction in peak principal stress (absolute) at or below +1.2 MPa and above −1.0 MPa and reduction in von Mises stresses below 1 MPa, with a lower, more uniform stress distribution during peak diastole. The polymeric leaflets have lower overall stresses while slightly reducing the overall volume of material in the polymeric leaflets (additionally benefiting the crimping stage). The same large opening area during systole is maintained with minimal differences in systolic stresses. This unique type of manufacturing of variable thickness polymeric leaflets is achievable with compression or injection molding, as two mating mold parts need to define the variable thickness throughout the entire leaflet surface.

### Validation of In Silico Models:

Interpretation and acceptance of the in silico models required validation of the results with in vitro models. The process of manufacturing each TAVR design iteration was not cost nor time effective, therefore the validation needed to be conducted at specific design points throughout the process. For example, the final stent design was cut and shape set, and radial force measurements were compared to the in silico results aiding in the acceptance of the crimped strain values previously obtained throughout the process. To validate the polymeric leaflet motion in the FEA models, the polymeric heart valve was manufactured and set up in a pulse duplicator (Left Heart Simulator, Vivitro Labs, Victoria, BC) and the motion was recorded with a high-speed camera (1057 FPS, Chronos 1.4, Kron Technologies Inc, Burnaby, BC). Due to the transparent polymeric leaflets, the raw images needed to be processed to enhance and extract the free edges, and the geometric orifice area (GOA, the projected opening area) can be obtained and compared to the in silico motion. There was excellent agreement between the opening area and profiles of the in vitro and in silico models, with minor discrepancies in the opening and closing speed. See FIG. 7.

### Superior Hydrodynamics:

The polymeric heart valve system in use is expected to have excellent performance in both idealized (Rotman, O. M., et al., Ann Biomed Eng, 47 (1), pp. 113-125) and patient-specific (Kovarovic, B. J., Rotman, O. M., Parikh, P., Slepian, M. J., and Bluestein, D., 2021, “Patient-specific in vitro testing for evaluating TAVR clinical performance-A complementary approach to current ISO standard testing,” Artif Organs, 45 (4), pp. E41-E52) in vitro testing scenarios.

### Superior Durability:

The instant polymeric heart valve system is an improvement of an earlier version. The prior generation system was tested with an Accelerated Wear Tester (AWT, HiCycle, Vivitro Labs, location) at the 10 Hz frequency, where the (n=4) prior generation valves have exceeded 900 M cycles with no loss in performance or noticeable wear and tear. A target of 1 billion cycles would represent an estimated 25 years of in vivo patient performance and is generally considered the target upper limit for valve devices (Sathananthan, J., et al., JACC: Cardiovascular Interventions, 13 (2), pp. 235-249). One notable feature of such testing is that the closing flow of the polymeric leaflets decreased over the testing lifetime as the free stress profile began to close. This reduction helped maintain the performance of the valve system by reducing the burden of the stroke volume. This opens the door to further exploit or utilize the fatigue of the polymeric leaflets to benefit the valve performance.

### Discussion:

The heart valve system described herein was designed with careful consideration and utilization of in silico modeling techniques to achieve a feasible and viable design.

The resulting stent (supportive) frame a yielded a manufacturable, repeatable stent design that meets or exceeds our expectations for the supportive frame. With incorporation of the sutureless overmold design, it was necessary to design for the additional material within the joint of each cell and to reduce the overall volume of the stent frame. The sutureless design showed adhesion between the polymer and nitinol, as well as ease of manufacture.

The polymeric leaflet design achieved lower stresses within the polymeric leaflets and reduced peak stresses at critical cycle time points. Reduction in the polymer volume within the leaflets helped reduce the crimping target size as well, potentially easing valve system delivery. Selection of the opening profile was used for stress reduction and was compatible with the molding technology.

### Conclusion

This example demonstrates a process for designing a polymeric TAVR device by utilizing in silico modeling of the various aspects of the device, from stent (supportive) frame crimping to the functioning of the leaflets in a typical cardiac cycle, while considering the features of the polymeric valve system designs. The polymeric leaflets were designed for a larger size device, further reducing the cyclic stresses.

1. A polymeric heart valve system comprising:
a supportive frame and a plurality of polymeric leaflets;
wherein the supportive frame comprises a plurality of openings, and wherein the supportive frame is embedded in a polymeric matrix in the form of a polymeric sleeve;
wherein each of the plurality of polymeric leaflets are a continuum of the polymeric sleeve without sutures, and each of the plurality of polymeric leaflets are connected to the polymeric sleeve at an attachment end; and
wherein the plurality of polymeric leaflets are able to open and close at an operative end, wherein when the plurality of leaflets are in the closed position, the operative ends of the leaflets abut each other.

2. The polymeric heart valve system of claim 1, wherein the supportive frame is formed from a plurality of connected struts each connected to one or more other struts via a joint.

3. (canceled)

4. (canceled)

5. The polymeric heart valve system of claim 1, wherein the supportive frame further comprises a crown region in which the polymeric matrix is not located, optionally wherein the crown region comprises one or more anchors.

6. (canceled)

7. The polymeric heart valve system of claim 1, wherein the supportive frame is made of a metal or metal alloy selected from the group consisting of spring steel, stainless steel, platinum, tantalum alloys, cobalt chromium, NiTi, NiTiCo, NiTiCr, NiTiCu, and NiTiNb.

8. (canceled)

9. The polymeric heart valve system of claim 1, wherein the polymeric sleeve comprises one or more polymers selected from the group consisting of thermoplastic polymers/elastomers, thermoset polymers, thermally crosslinkable polymers, elastomeric polymer biomaterials, polymers containing polysulfone(s), PTFE, crosslinked poly (styrene-isobutylenese-styrene) (xSIBS), poly (styrene-isobutylene-styrene) (SIBS), polymyrcene, polymenthide, and poly (ϵ-decalactone), silicones, polyolefins, polydiene elastomers, poly (vinyl chloride), natural rubbers, heparinized polymers, hydrogels, polypeptides elastomers, polysiloxane-urea elastomers, and polyurethanes.

10. The polymeric heart valve system of claim 1, wherein the polymeric sleeve further comprises one or more anti-leak flaps thereon.

11. (canceled)

12. (canceled)

13. The polymeric heart valve system of claim 1, wherein the polymeric leaflets comprise one or more polymers selected from the group consisting of thermoplastic polymers/elastomers, thermoset polymers, thermally crosslinkable polymers, elastomeric polymer biomaterials, polymers containing polysulfone(s), PTFE, crosslinked poly (styrene-isobutylenese-styrene) (xSIBS), poly (styrene-isobutylene-styrene) (SIBS), polymyrcene, polymenthide, and poly (ϵ-decalactone), silicones, polyolefins, polydiene elastomers, poly (vinyl chloride), natural rubbers, heparinized polymers, hydrogels, polypeptides elastomers, polysiloxane-urea elastomers, and polyurethanes.

14. The polymeric heart valve system of claim 1, wherein each of the leaflets in the plurality of leaflets comprises variable thickness having a center line of symmetry, where in a given cross-section each of the leaflets has at least two or more thicknesses.

15. The polymeric heart valve system of claim 14, wherein each of the leaflets has a minimum thickness in a range from about 50 to 200 μm or in a range from about 200 to 600 μm.

16. (canceled)

17. (canceled)

18. (canceled)

19. A method of making a polymeric heart valve system, the method comprising the steps of:
(i) placing a supportive frame inside a cavity of a mold in a position suitable to facilitate flow of the polymer around the supportive frame,
wherein the mold comprises a top core, a bottom core, and one or more encasement components which encase the top and bottom cores,
wherein the top core and bottom core define a shape to form polymeric leaflets in a suitable orientation wherein the polymeric leaflets are formed preferably in a semi-open position having a zero residual stress conformation,
optionally, wherein the shape forming the polymeric leaflets has a curvilinear profile along a circumferential axis of the polymeric leaflets being formed; and
(ii) introducing at least one polymer into the mold;
(iii) molding or casting the polymeric leaflets and a polymeric sleeve around the supportive frame to form a polymeric heart valve system,
wherein the polymeric leaflets and the polymeric sleeve are a continuum and each of the polymeric leaflets are connected to the polymeric sleeve at an attachment end; and
(iv) removing the polymeric heart valve system formed from the mold.

20. The method of claim 19, wherein the one or more encasement components further comprising one or more arched segments or pins and/or one or more sealing elements;
wherein the one or more arched segments or pins hold the supportive frame.

21. (canceled)

22. (canceled)

23. (canceled)

24. (canceled)

25. (canceled)

26. (canceled)

27. The method of claim 19, wherein the supportive frame is formed from a plurality of connected struts each connected to one or more other struts via a joint.

28. (canceled)

29. The method of claim 19, wherein the supportive frame is made of a metal or metal alloy selected from the group consisting of spring steel, stainless steel, platinum, tantalum alloys, cobalt chromium, NiTi, NiTiCo, NiTiCr, NiTiCu, and NiTiNb.

30. The method of claim 19, wherein the polymer is selected from one or more of the group consisting of thermoplastic polymers/elastomers, thermoset polymers, thermally crosslinkable polymers, elastomeric polymer biomaterials, polymers containing polysulfone(s), PTFE, crosslinked poly (styrene-isobutylenese-styrene) (xSIBS), poly (styrene-isobutylene-styrene) (SIBS), polymyrcene, polymenthide, and poly (ϵ-decalactone), silicones, polyolefins, polydiene elastomers, poly (vinyl chloride), natural rubbers, heparinized polymers, hydrogels, polypeptides elastomers, polysiloxane-urea elastomers, and polyurethanes.

31. The method of claim 19, wherein the polymeric sleeve further comprises one or more anti-leak flaps thereon.

32. (canceled)

33. The method of claim 19, wherein each of the polymeric leaflets comprises variable thickness across cross-sections of each of the polymeric leaflets running from an attachment end to an operative end thereof.

34. The method of claim 33, wherein each of the polymeric leaflets has a minimum thickness in a range from about 50 to 200 μm or in a range from about 200 to 600 μm.

35. (canceled)

36. (canceled)

37. A polymeric heart valve system comprising:
a supportive frame and a plurality of polymeric leaflets;
wherein the supportive frame comprises a plurality of openings, and wherein the supportive frame is embedded in a polymeric matrix in the form of a polymeric sleeve;
wherein each of the plurality of polymeric leaflets are a continuum of the polymeric sleeve without sutures, and each of the plurality of polymeric leaflets are connected to the polymeric sleeve at an attachment end; and
wherein the plurality of polymeric leaflets are able to open and close at an operative end, wherein when the plurality of leaflets are in the closed position, the operative ends of the leaflets abut each other;
wherein the polymeric heart valve system is formed according to the method of claim 19.

38. A method of replacing a defective valve in a subject in need thereof, the method comprising the steps of:
(1) inserting a polymeric heart valve system of claim 1 in a crimped, collapsed, or compacted state into the subject in need thereof through an artery;
(2) delivering the polymeric heart valve system to the defective valve in the subject in need thereof;
(3) implanting the polymeric heart valve system by expanding the crimped, collapsed, or compacted polymeric heart valve system into an expanded state, which localizes or fixes the polymeric heart valve system at the defective valve and replaces the function of the defective valve.

39. The method of claim 38, wherein the defective valve is an aortic valve.

40. (canceled)

41. (canceled)

42. (canceled)

43. (canceled)
